(19) United States
(12) Patent Application Publication
(10) Pub. No.: US 2013/0210143 A1
(43) Pub. Date: Aug. 15, 2013

(54) NOVEL METHODS FOR PREVENTING OR TREATING DIABETES

(71) Applicants: Luc St-Onge, Neuried (DE); Benoit R. Gauthier, Seville (ES); Mathurin Baquie, Toulouse (FR); Claes B. Wollheim, Vessy (CH)

(72) Inventors: Luc St-Onge, Neuried (DE); Benoit R. Gauthier, Seville (ES); Mathurin Baquie, Toulouse (FR); Claes B. Wollheim, Vessy (CH)

(73) Assignees: FUNDACION PUBLICA ANDALUZA PROGRESO Y SALUD, Sevilla (ES); Luc ST-ONGE, Neuried (DE)

(21) Appl. No.: 13/699,184

(22) PCT Filed: May 20, 2011
(86) PCT No.: PCT/EP2011/058232

§ 371 Date: Apr. 8, 2013

(30) Foreign Application Priority Data
May 20, 2010 (EP) 10005286.9

(51) Int. Cl. C12N 5/071

(52) U.S. Cl. CPC C12N 5/0676
USPC 435/374

(57) ABSTRACT

The present application relates to an LRH-1 agonist for use in the prevention of progressive loss of pancreatic β-cells. It also relates to an LRH-1 agonist for use in the preservation or restoration of pancreatic β-cells. Further, it relates to an LRH-1 agonist for use in the prevention or treatment of type I diabetes, the increment of survival of pancreatic β-cells, the increment of the performance of pancreatic β-cells, the increment of the survival of a β-cell graft, the in vitro preservation of pancreatic β-cells, maintaining insulin secretion and/or in a method of transplanting pancreatic islet cells.

The present application relates to an LRH-1 agonist for use in the prevention of progressive loss of pancreatic β-cells. It also relates to an LRH-1 agonist for use in the preservation or restoration of pancreatic β-cells. Further, it relates to an LRH-1 agonist for use in the prevention or treatment of type I diabetes, the increment of survival of pancreatic β-cells, the increment of the performance of pancreatic β-cells, the increment of the survival of a β-cell graft, the in vitro preservation of pancreatic β-cells, maintaining insulin secretion and/or in a method of transplanting pancreatic islet cells.

Diabetes mellitus (DM), often simply referred to as diabetes, is a major global health burden with 3.2 million deaths per year and six deaths attributable to diabetes or related conditions every minute (Roglic, 2005). Diabetes is a condition in which a person has a high blood sugar (glucose) level as a result of the body either not producing enough insulin, or because body cells do not properly respond to the insulin that is produced.

In healthy persons, blood glucose levels are maintained within a narrow range, primarily by the actions of the hormone insulin. Insulin is released by pancreatic β-cells at an appropriate rate in response to circulating glucose concentrations, the response being modulated by other factors including other circulating nutrients, islet innervation and incretin hormones. Insulin maintains glucose concentrations by constraining the rate of hepatic glucose release to match the rate of glucose clearance.

Insulin thus enables body cells to absorb glucose, to turn into energy. If the body cells do not absorb the glucose, the glucose accumulates in the blood (hyperglycemia), leading to various potential medical complications. Accordingly, diabetes is characterized by increased blood glucose resulting in secondary complications such as cardiovascular diseases, kidney failure, retinopathy and neuropathy if not properly controlled (Calcutt, (2009). Two major pathophysiologies are related to increase glycemia. The first is an autoimmune attack against the pancreatic insulin-producing β-cells (Type 1 diabetes) whilst the second is associated to poor β-cell function and increased peripheral insulin resistance (Type 2 diabetes). Similar to Type 1, β-cell death is also observed in Type 2 diabetes. Type 1 and often Type 2 diabetes requires the person to inject insulin.

Type 1 DM is typically characterized by loss of the insulin-producing β-cells of the islets of Langerhans in the pancreas leading to insulin deficiency. This type of diabetes can be further classified as immune-mediated or idiopathic. The majority of Type 1 diabetes is of the immune-mediated nature, where β-cell loss is a T-cell mediated autoimmune attack. There is no known preventive measure against Type 1 diabetes, which causes approximately 10% of DM cases in North America and Europe. Most affected people are otherwise healthy and of a normal weight when onset occurs. Sensitivity and responsiveness to insulin are usually normal, especially in the early stages. Type 1 diabetes can affect children or adults but was traditionally termed “juvenile diabetes” because it represents a majority of the diabetes cases in children.

Type 2 DM is characterized by β-cell dysfunction in combination with insulin resistance. The defective responsiveness of body tissues to insulin is believed to involve the insulin receptor. Similar to Type 1 diabetes an insufficient beta cell mass is also a pathogenic factor in many Type 2 diabetic patients. In the early stage of Type 2 diabetes, hyperglycemia can be reversed by a variety of measures and medications that improve insulin secretion and reduce glucose production by the liver. As the disease progresses, the impairment of insulin secretion occurs, and therapeutic replacement of insulin may sometimes become necessary in certain patients

However, though both forms of diabetes have been treatable since insulin became medically available in 1921, a therapy is nevertheless difficult. Pancreas transplants have been tried with limited success in Type 1 diabetes; gastric bypass surgery has been successful in many with morbid obesity and Type 2 diabetes; and gestational diabetes usually resolves after delivery. Diabetes without proper treatments can cause many complications. Acute complications include hyperglycaemia, diabetic ketoacidosis, or nonketotic hyperosmolar coma. Serious long-term complications include cardiovascular disease, chronic renal failure, retinal damage.

In sum diabetes is a frequent chronic disease that can appear at all ages. It reduces quality of life and increases the risk for life threatening complications despite current treatment. Its economic burden is estimated at 15% of health care expenses. As both types of diabetes are, inter alia, characterized by the loss of β-cell mass due to cell death, the restoration and preservation of the β-cell mass is a major goal as it may cure diabetes.

Accordingly, novel treatment should therefore aim at reducing beta-cell death in the attempt to maintain a sufficient critical functional β-cell mass that can normalize blood glucose.

Hence, given the above, it would be desirable to restore or preserve β-cell mass in order to prevent or treat diabetes or, put in other words, prevent the progressive loss of β-cell mass.

Accordingly, the technical problem of the present invention is to comply with the needs described above.

The present invention addresses this need and thus provides as a solution to the technical problem methods and compositions for use in the prevention of progressive loss of pancreatic β-cells or, put in other words, methods for the preservation of pancreatic β-cells, thereby making available novel methods for the prevention or treatment of diabetes. These embodiments are characterized and described herein, shown in the Figures, illustrated in the Examples, and reflected in the claims.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

All publications and patents cited in this disclosure are incorporated by reference in their entirety. To the extent the material incorporated by reference contradicts or is inconsistent with this specification, the specification will supersede any such material.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

In view of the reduced β-cell mass in Type 2 diabetes and destruction of cells in Type 1 diabetes, the identification of genes conferring replicative and/or protective properties was the starting point for the present inventors who aimed for the development of cell regeneration therapy.

Accordingly, the present inventors identified that LRH-1 is such a target gene. Specifically, the inventors determined that LRH-1 is expressed in the endocrine pancreas. More specifically, they show that rat and human islet β-cells express LRH-1.

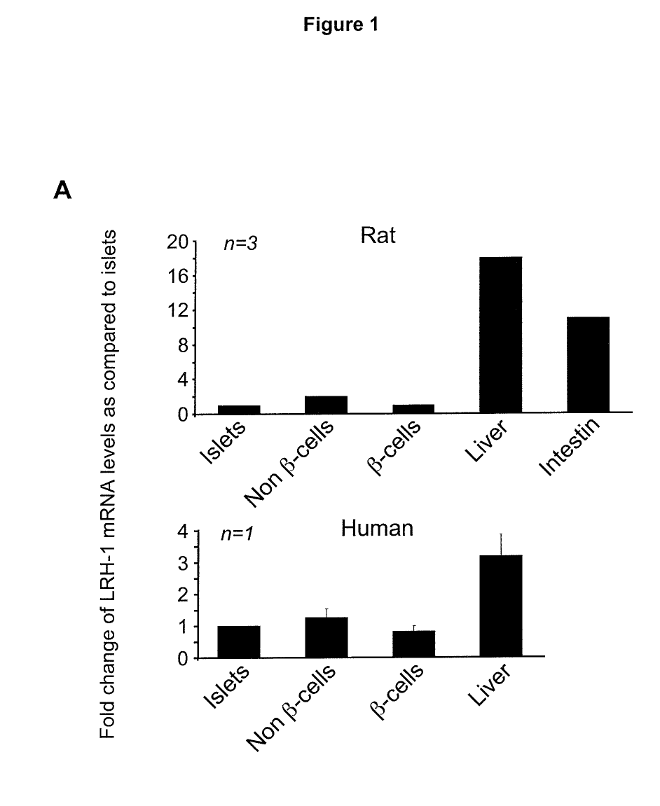

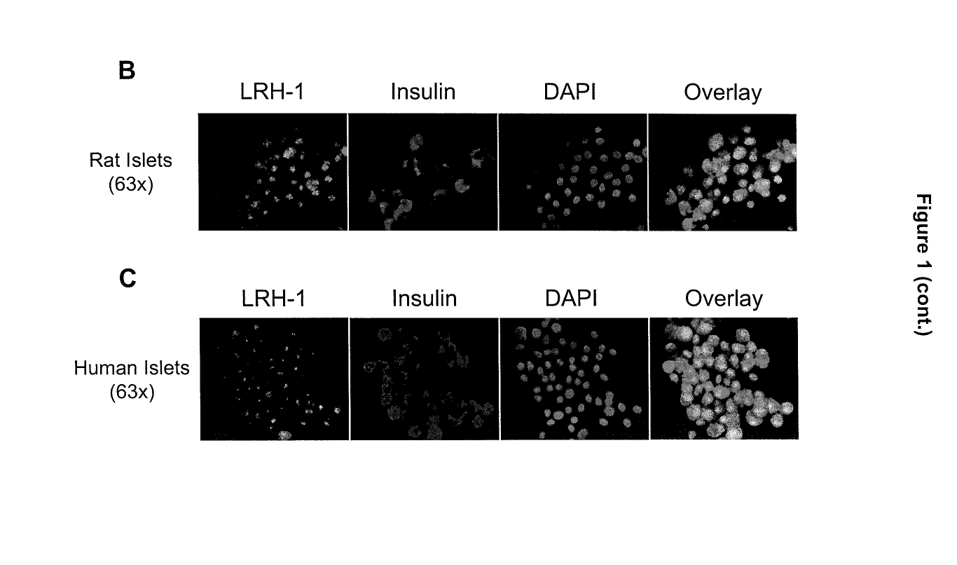

In detail, the inventors conducted quantitative RT-PCR and immunofluorescent studies on rat and human islets. It was found that LRH-1 was expressed in mature rodent and human islets of Langerhans as well as in FACS purified β- and non β-cells of these islets, albeit at lower levels than those detected in the control liver and intestine samples (FIG. 1A). LRH-1 transcript levels appeared more abundant in the non β-cell fraction as compared to purified β-cells. Interestingly, rat and human islets expressed similar levels of the nuclear orphan receptor whereas transcript levels were 6-fold higher in rat liver as compared to the human organ.

Co-immunofluorescence analysis performed using anti-LRH-1 and anti-insulin serum confirmed the presence of endogenous LRH-1 in β- as well as non β-cells (FIG. 1B). In rat islets, the staining was dispersed throughout the nuclei and cytoplasm whereas in human islets LRH-1 predominantly stained in sub-regions of the nuclei. These results clearly demonstrate that the nuclear receptor is expressed in rodent and human pancreatic islets, including β-cells.

In a next step, the inventors evaluated the potential impact of LRH-1 on β-cell physiology.

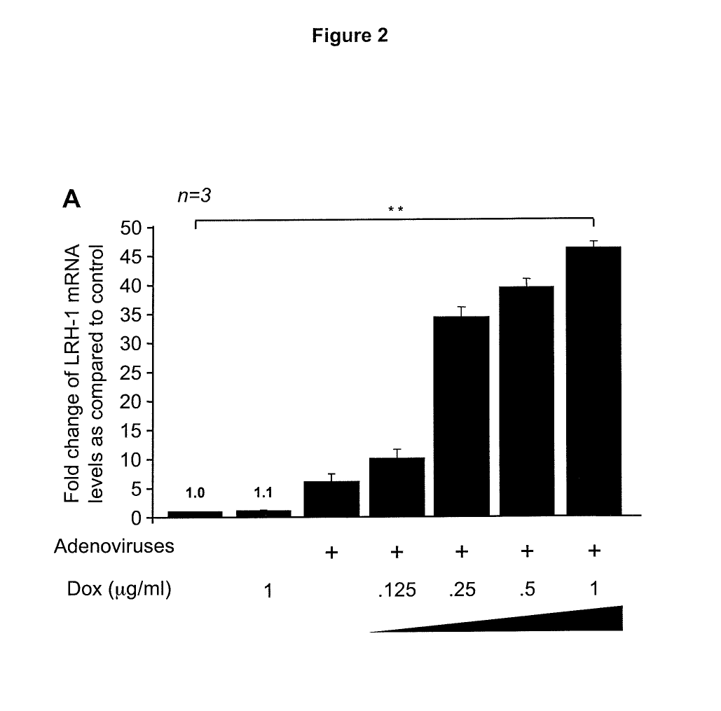

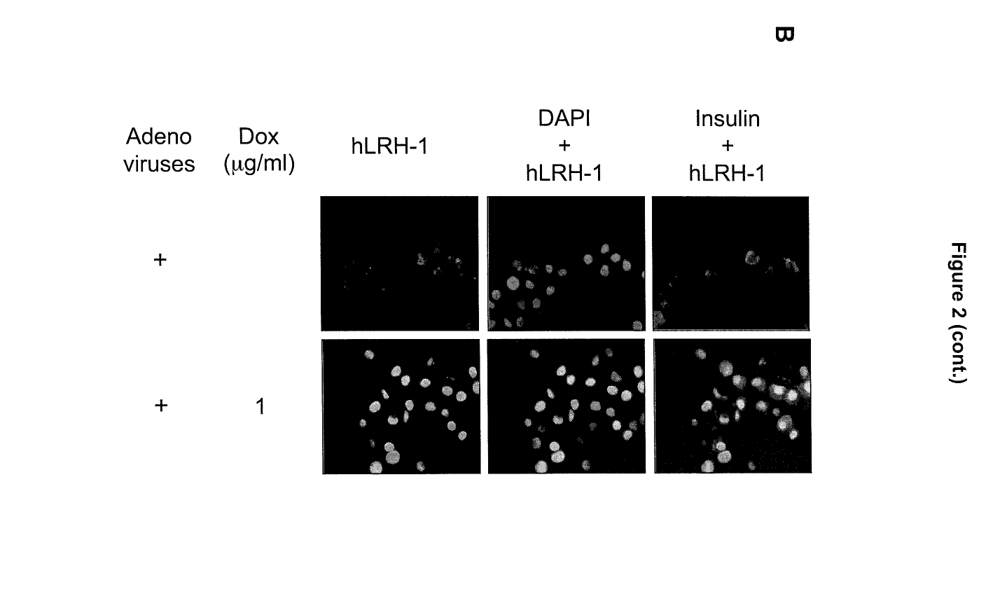

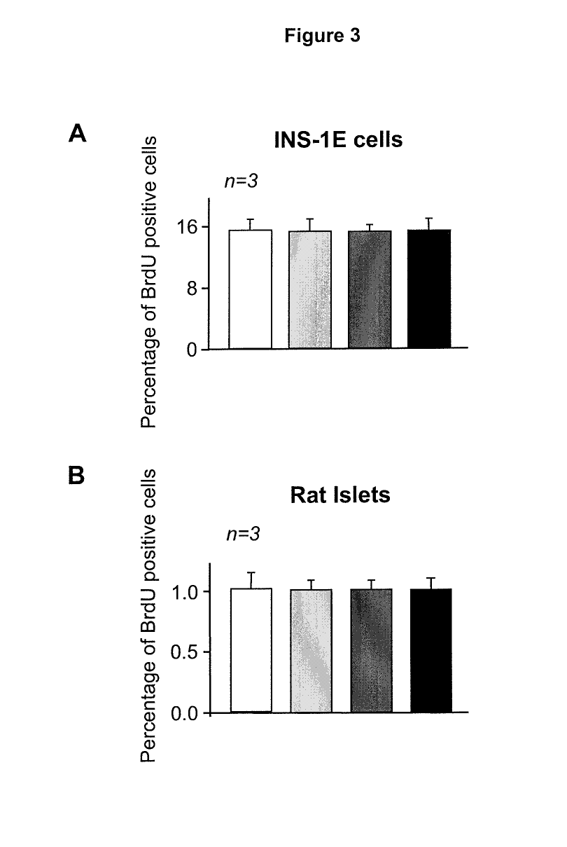

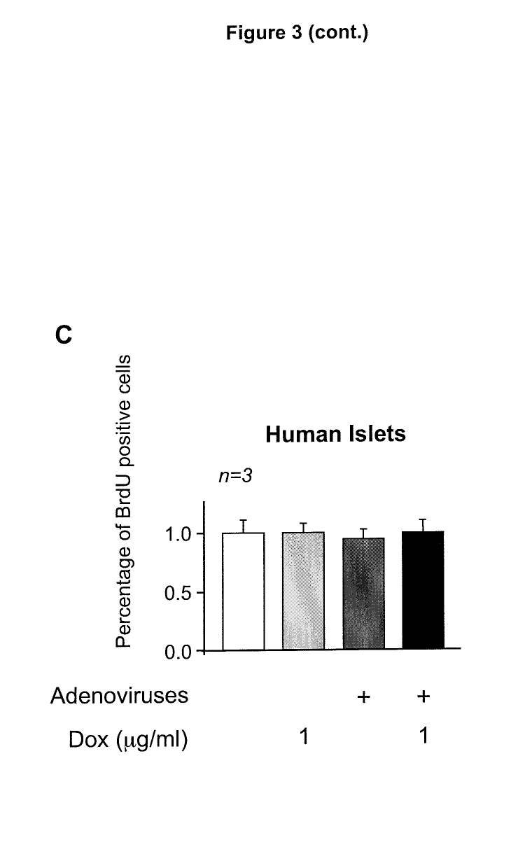

They observed surprisingly that adenoviral mediated over expression of LRH-1 protects insulin-producing β-cells from cell death (apoptosis) promoting agents such as cytokines. In particular, in the attempt to gain insight into the functional impact of LRH-1 on β-cells, LRH-1 was conditionally over expressed in islets using tetracycline inducible adenoviruses. Isolated human islets were infected with Ad-hLRH-1 and incubated for 48 h in the presence or absence of increasing concentrations of the inducing tetracycline analogue, doxycycline. Quantitative RT-PCR established a dose-dependent stimulation in LRH-1 expression levels, reaching a 48-fold increase as compared to control untreated islets at 1 μg/ml doxycycline (FIG. 2A). A strong nuclear immunostaining was detected in 80% of human islet cells treated with doxycycline while a diffused nuclear and cytoplasmic staining was observed in control islets (FIG. 2B). Similar results were obtained in rat islets as well as in the insulinoma INS-1E cell line. Moreover, over-expression of LRH-1 did not alter cell proliferation in INS-1E cells, rat islets or human islets as compared to matched control (FIG. 3).

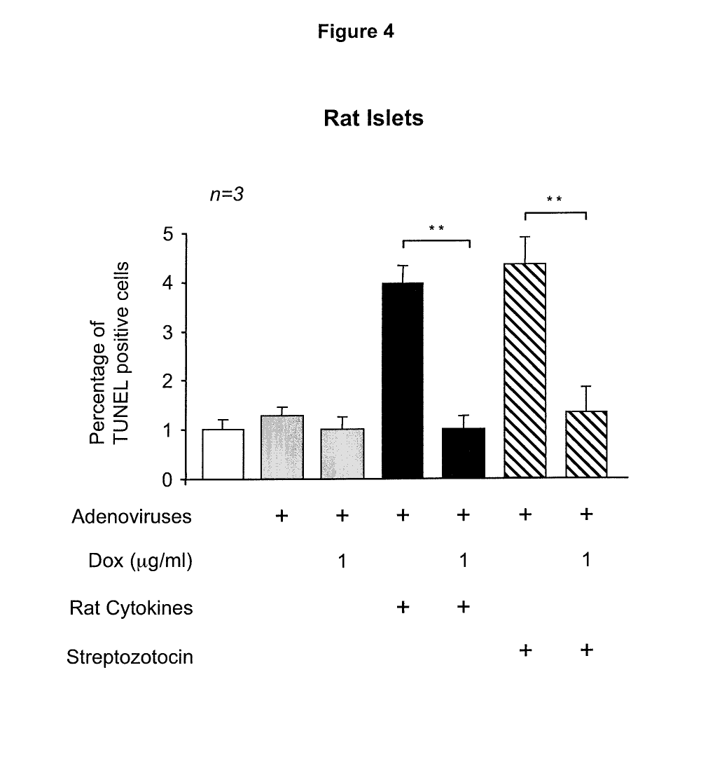

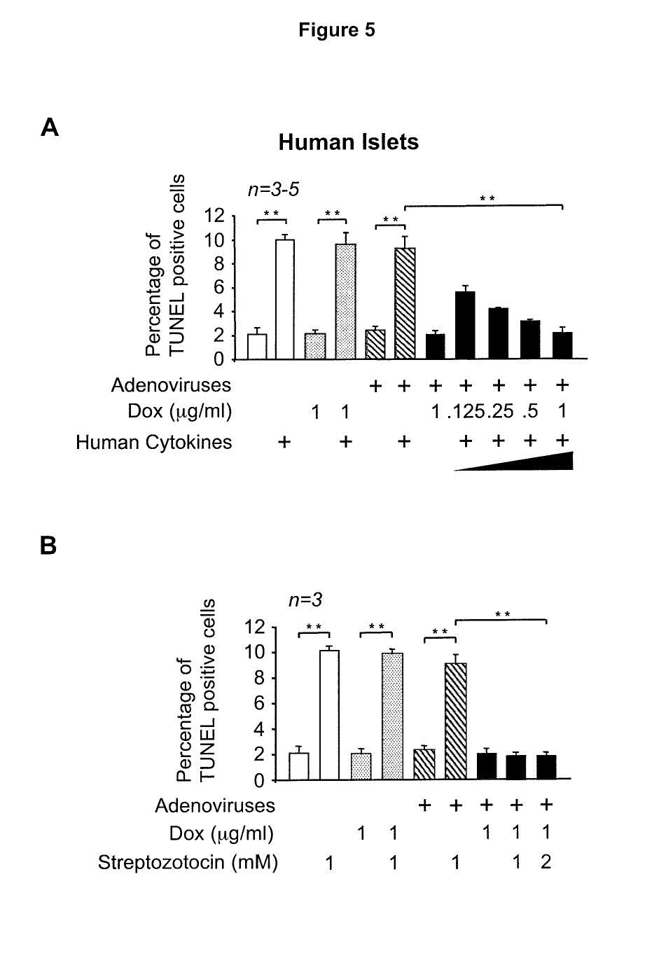

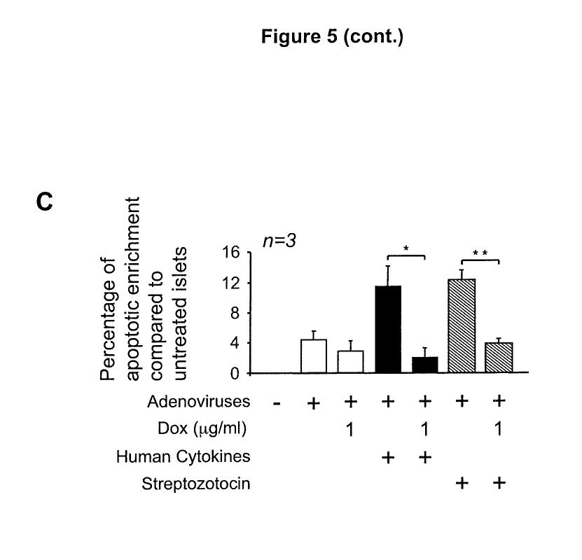

Further, the inventors evaluated the potential protective role of LRH-1 over expression in rat and human islets exposed to either cytokines or streptozotocin. A 4-fold increase in TUNEL-positive cells was estimated in control rat islets cultured in the presence of cytokines or streptozotocin. However, doxycycline-induced LRH-1 expression completely protected islets against apoptosis (FIG. 4). Similarly, human islets exhibited a 10-fold increased in cytokine-mediated apoptosis, an effect that was dose dependently attenuated by increasing concentrations of doxycycline (FIG. 5A). LRH-1 over expressing human islets were also refractory to apoptosis induced by elevated doses of streptozotocin (FIG. 5B). As the TUNEL assay may be prone to false-positive results, apoptosis was alternatively measured by using a cell death detection ELISA system. To this end identical results were obtained (FIG. 5C). Taken together these results clearly suggest that LRH-1 is involved in cell survival and that over expressing LRH-1 in rat and human islets protects these cells against cytokine-induced apoptosis.

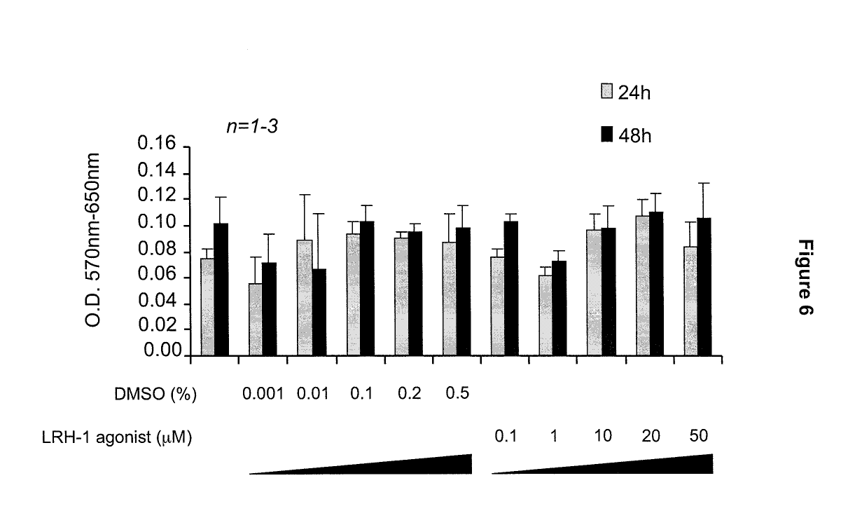

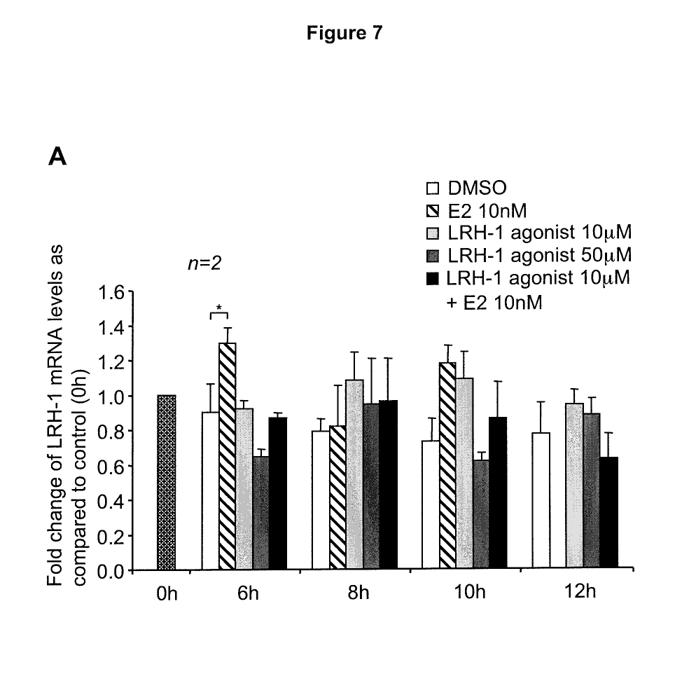

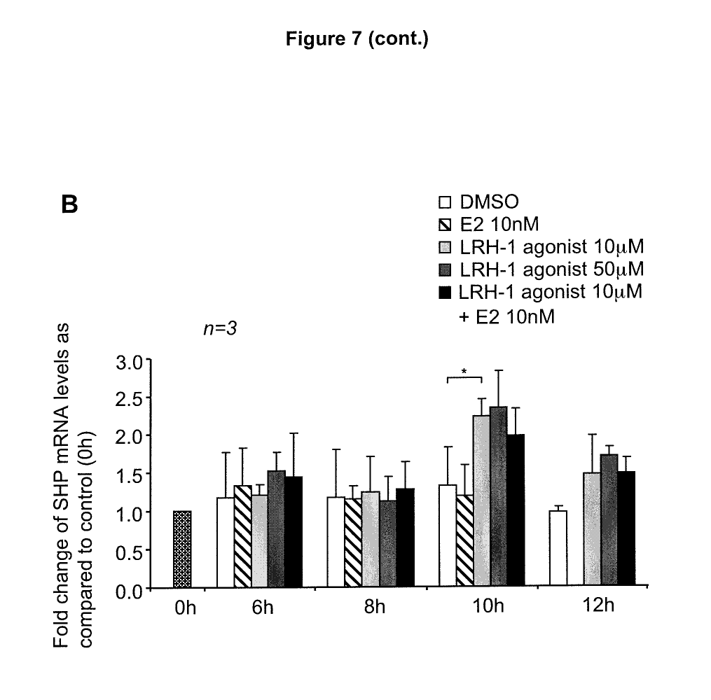

Even more surprisingly they observed that treatment of human islets with a synthetic 9-substituted bicycle [3.3.0] octane derivative such as a substituted cis-bicyclo[3.3.0]-oct-2-ene compound that is an agonist of LRH-1 (described herein below), prevented cell death induced by cytokines and stimulated insulin secretion in islets isolated from a Type 2 diabetic donor. Indeed, after having shown that an exemplary LRH-1 agonist is not toxic to islets (FIG. 6), the inventors demonstrated that this exemplary LRH-1 agonist activates the nuclear orphan receptor (FIGS. 7A and B).

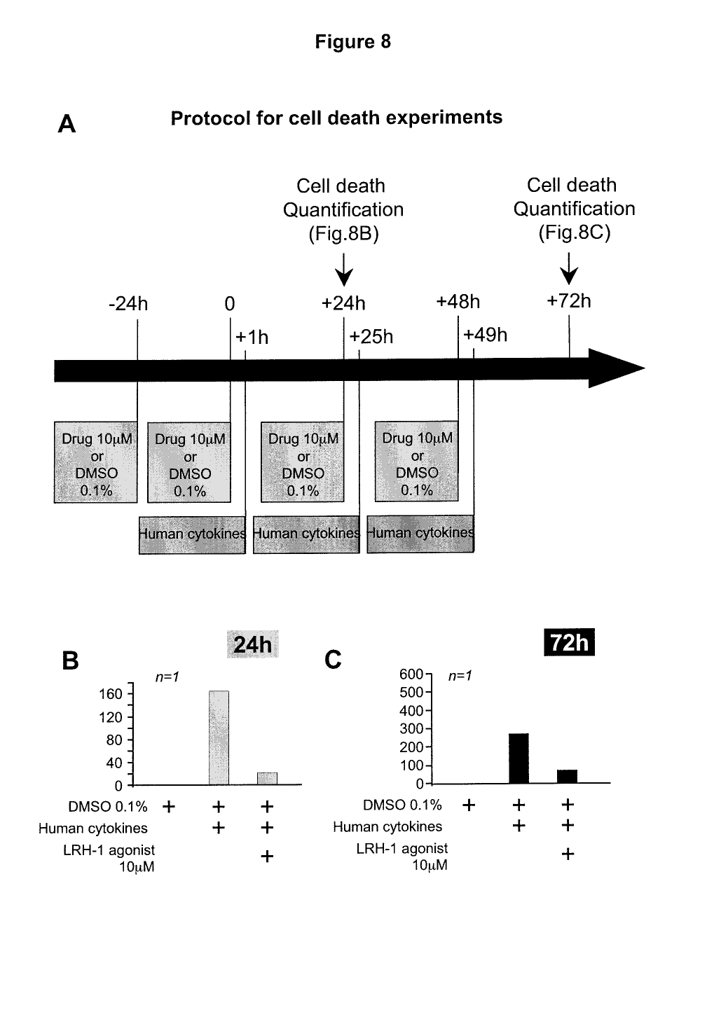

Moreover, the inventors showed that an exemplary 9-substituted bicycle [3.3.0] octane derivative such as a substituted cis-bicyclo[3.3.0]-oct-2-enes that is a LRH-1 agonist protects human islets from cytokine-induced apoptosis. Next, the capacity of a LRH-1 agonist to preserve islet cells against stress-induced apoptosis was evaluated. To this end, human islets were treated with 4 consecutive doses of a LRH-1 agonist or 0.1% DMSO at intervals of 24 hours (−24, 0, 24 and 48 hours). In addition, islets were also treated with 3 doses of cytokines starting 25 hours after the initial drug treatment (1, 25 and 49 hours). Apoptosis was then measured at 24 and 48 hours (FIG. 8A). Cytokine-treated islets exhibited 160% enrichment in apoptotic cells as compared to control untreated islets 24 hours post-treatment (FIG. 8B). Remarkably this effect was completely abrogated in islets cultured in the presence of 10 μM of a LRH-1 agonist (FIG. 8B). A similar beneficial effect was observed in cytokines treated islets at 72 hours post treatment (FIG. 8C). Taken together, these results demonstrate that activation of endogenous LRH-1 using an LRH-1 agonist can confer protection against cytokine-induced apoptosis to human islets.

Accordingly, given the observations made and the results obtained by the inventors, it is apparent that the increase of the expression or activity of LRH-1 has a positive effect on insulin-producing β-cells in that the cells are protected from cell death inducing agents which play a major role in the onset and aetiology of diabetes.

Hence, in a first aspect, the present invention relates to an LRH-1 agonist for use in the prevention of progressive loss of pancreatic β-cells, preferably pancreatic islet β-cells.

Any of the uses and methods applying a LRH-1 agonist is preferably for use in a subject. A “subject” when used herein includes mammalian and non-mammalian subjects. “Mammal” for purposes of treatment refers to any animal classified as a mammal, including human, domestic and farm animals, nonhuman primates, and any other animal that has mammary tissue. A mammal includes human, rodents such as mouse, rat or rabbit, sheep, cattle, goat, dog, cat, chimpanzee, horse, pig, etc., with human being preferred. A subject also includes human and veterinary patients, with human patients being preferred.

A “patient” is an individual subject having a progressive loss of pancreatic β-cells. The term “patient” also includes an “individual suspected of having a progressive loss of pancreatic β-cells”.

LRH-1 (liver receptor homolog-1) is a member of the NR5A2 or Ftz-F1 subfamily of nuclear receptors that binds as a monomer to its target genes. This nuclear receptor plays a pivotal role in endodermal development by controlling expression of key transcription factors such as Foxa2, HNF-4α and HNF-1α (Rausa, (1997); Pare, (2001)).

Interestingly, LRH-1 gene expression is regulated by the pancreatic-duodenal homeobox 1 (Pdx1) factor during pancreas development (Annicotte, (2003)). In adult mammals, the nuclear receptor is predominantly expressed in the liver, exocrine pancreas, intestine and ovaries. Interestingly, LRH-1 is the most abundant nuclear receptor expressed in human pancreatic islets, in particular, in α-cells. (Chuang (2008)).

LRH- has sequence similarity to SF-1. Gene knockout and heterozygous loss-of-function studies show that both SF-1 and LRH-1 are essential during embryogenesis for normal development of the organs in which they are expressed, and mammalian cell transfection experiments indicate that SF-1 and LRH-1 function as obligate factors for their target genes, acting apparently constitutively.

In particular, LRH-1 regulates expression of genes involved in cholesterol and bile acid metabolism as well as steroidogenesis (Lee et al. (2008)). In particular, LRH-1 is involved in the regulation of a number of different genes, including, for example, steroidogenic acute regulatory protein (Kim et al. (2004), J. Clin Endocrinol Metab. 89:3042-3047), apolipoprotein Al (Delerive et al. (2004), Mol. Endocrinol. 18:2378-87), cholesterol 7 alpha-hydroxylase (Qin et al. (2004), Mol. Endocrinol. 18:2424-2439), aromatase (Clyne et al. (2004), Mol. Cell. Endocrinol. 215:39-44), carboxyl ester lipase (Fayard et al. (2003)), and cytochrome P450 7A.

Although LRH-1 is considered an orphan receptor that possesses constitutive activity, small phospholipids such as phosphatidylethanolamine were shown to occupy the ligand pocket domain and to increase LRH-1 activity (Ortlund et al. (2005), Nat. Struct. Mol. Biol. 12:35-363). In addition, small bicyclic compounds were recently found to be efficient agonists of LRH-1 activity (Whitby et al. (2006)). Interestingly, the LRH-1 downstream target gene SHP (small heterodimer partner), binds to and serves as a potent co-repressor of LRH-1 (Ortlund et al. (2005)).

Furthermore, LRH-1 was shown to contribute to intestinal and gastric tumour formation (Schoonjans, (2005)); Wang, (2008)) as well as to promote cell proliferation in murine pancreatic and hepatic cell lines (Botrugno, (2004)). Suppression of LRH-1 by RNA interference provoked apoptosis in the hepatocellular carcinoma cell line BEL-7402 (Wang et al. (2005)). Consistent with its oncogenic properties, LRH-1 was also found to mediate the 17β-estradiol/estrogen receptor α (ER α)-dependent proliferation of the MCF7 breast cancer cell line (Annicotte, (2005)). LRH-1 would also appear to have a cell protective role by inhibiting cytokine-elicited inflammatory responses in the liver (Venteclef, (2006)). Interestingly, isolated human islets treated with 17 β-estradiol were protected against proinflammatory cytokine-induced cell death (Contreras, (2002)). Furthermore, ER a deficient mice, independent of gender, were found to be more susceptible to islet β-cell apoptosis and prone to insulin-deficient diabetes subsequent to acute oxidative stress (Le May, (2006)).

As a typical nuclear receptor LRH-1 contains a ligand binding domain (LBD). Ligand binding induces the activation function-2 helix of the LBD to form a charge clamp for co-activator recruitment (Feng et al. (1998), Science 280:1747-1749). The LRH-1 LBD is composed of two short β-strands and 12 α-helices arranged in a four-layer helical sandwich with an extended helix 2 forming the additional layer. LRH-1 is known to interact with typical transcriptional co-regulators like GRIP-1 and SRC-1. It also interacts with AF-2, yet the AF-2 surface adjacent to LRH-1's helix 12, α-AF, does not seem to be optimized for this protein. However, the small heterodimer partner (SHP), an atypical nuclear receptor lacking a DNA binding domain, serves as an efficient and potent co-repressor of LRH-1's action in numerous tissues (Ortlund et al. (2005), Nat. Struct. Mol. Biol. 12:35-363).

As used herein, the terms “liver receptor homolog 1 ligand binding domain polypeptide”, “LRH-1 ligand binding domain polypeptide”, and “LRH-1 LBD polypeptide” (and like terms) refer to a polypeptide that contains the site where phospholipid binding as identified herein occurs. For human LRH-1, such domain generally includes residues A253 through A495 of NP_{—}003813 encoded by NM_{—}003822. For mouse LRH-1, such sequence generally extends from A318 through A560 of the protein encoded by NM_{—}030676. An exemplary such human domain polypeptide is the polypeptide used for crystallization herein consisting of residues S251-A495 of NP_{—}003822 (see US 2006/0160135); additional examples include homologs and variants thereof.

When used in the context of the present application the term “LRH-1” includes a LRH-1 (also known as NR5A2, CPF or FTF) polynucleotide or polypeptide having a nucleotide or amino acid sequence, respectively, as is known in the art; see NM_{—}003822 (cDNA sequence for hLRH-1 isoform 2), NP_{—}003813 (protein sequence for hLRH-1 isoform 2), NM_{—}205860 (cDNA sequence for hLRH-1 isoform 1), and NP_{—}995582 (protein sequence for hLRH-1 isoform 1). When referring herein to LRH-1 nucleotide sequences or LRH-1 amino acid sequences, the afore-mentioned sequences are preferred as “reference sequences” when, e.g. determining the degree of identity of nucleotide or amino acid sequences which are encompassed by the term “LRH-1”. The term “LRH-1” also includes nucleotide sequences which are 60, 70, 80, 90, 95, 97, 98, 99% identical to the LRH-1 nucleotide sequences which are known in the art and described herein, wherein these 60, 70, 80, 90, 95, 97, 98, 99% identical nucleotide sequences encode a LRH-1 polypeptide which retains the activity as described herein. The nucleotide sequences according to the invention may be any type of nucleic acid, e.g. DNA, RNA or PNA (peptide nucleic acid).

The term “LRH-1” also includes amino acid sequences which are 60, 70, 80, 90, 95, 97, 98, 99% identical to the LRH-1 amino acid sequences which are known in the art and described herein, wherein these 60, 70, 80, 90, 95, 97, 98, 99% identical amino acid sequences retain the activity of LRH-1 as described herein. Said term also includes LRH-1 polypeptide variants having an amino acid sequence, wherein in such variants one or more, preferably 2, 4, 6, 8, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 510, 520, 530 or 540 amino acids are added, deleted and/or substituted as long as such LRH-1 polypeptide variants retain the activity as described herein. Said term also includes LRH-1 polypeptide fragments, preferably of 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 510, 520, 530 or 540 amino acids in length, wherein such fragments retain the activity as described herein.

LRH-1 homologs which are also included by the term “LRH-1” can be identified by their sequences, where exemplary reference sequence accession numbers are NM_{—}003822 (cDNA sequence for hLRH-1 isoform 2), NP_{—}003813 (protein sequence for HLRH-1 isoform 2), NM_{—}205860 (cDNA sequence for hLRH-1 isoform 1), and NP_{—}995582 (protein sequence for hLRH-1 isoform 1). One of ordinary skill in the art will recognize that sequence differences will exist due to allelic variation, and will also recognize that other animals, particularly other mammals, have corresponding receptors, which have been identified or can be readily identified using sequence alignment and confirmation of activity, which can also be used. A number of such sequences are readily available from GenBank. One of ordinary skill in the art will also recognize that modifications can be introduced in a LRH-1 sequence without destroying receptor activity. Such modified receptors can also be used in the present invention, e.g., if the modifications do not alter the binding site conformation to the extent that the modified receptor lacks substantially normal ligand binding.

The term “LRH-1 agonist” means an agent or a compound that increases, supplements, or potentiates the bioactivity of LRH-1. Preferably, an LRH-1 agonist interacts directly or indirectly with LRH-1 and initiates a physiological and/or a pharmacological response characteristic of LRH-1. An LRH-1 agonist is believed to increase, supplement, or potentiate the activity of LRH-1 or the expression of LRH-1 either directly or indirectly. The action of an LRH-1 agonist can, for example, occur at the protein level. Particularly, LRH-1 may interact with the agonist such that it is more active. The action of an LRH-1 agonist may, however, also occur on nucleic acid level. Namely, the LRH-1 gene is transcribed more frequently giving rise to more protein. The action of an LRH-1 agonist may also influence RNA or protein stability. A direct interaction via binding of the agonist to LRH-1 is, however, preferred. Though less preferred, it is nevertheless envisaged that an LRH-1 agonist may act as an agonist of SF-1, i.e., act as an SF-1 agonist.

By way of interacting with LRH-1, an LRH1 agonist as applied in the embodiments of the invention may also increase coactivator (co-regulator) peptide recruitment to the ligand binding domain. Exemplary coactivators of LRH-1 are GRIP-1, SRC-1 or AF-2.

As used herein in connection with the interaction of the agonist with LRH-1, the term “bind” and “binding” and like terms refer to a non-covalent energetically favourable association between the agonist and LRH-1 (i.e., the bound state has a lower free energy than the separated state, which can be measured calorimetrically). For binding to a target, the binding is at least selective, that is, the agonist binds preferentially to LRH-1 and/or SF-1 with LRH-1 being preferred, but not to other members of the NR52A family at a binding site, as compared to non-specific binding to unrelated proteins not having a similar binding site. For example, BSA is often used for evaluating or controlling non-specific binding. In addition, for an association to be regarded as binding, the decrease in free energy going from a separated state to the bound state must be sufficient so that the association is detectable in a biochemical assay suitable for the molecules involved.

Preferably, as “agonist”, in accordance with this invention, molecules/substances are denoted which have an affinity as well as an intrinsic activity. Mostly, said intrinsic activity (α) is defined as being proportional to the quotient of the effect, triggered by said agonist (EA) and the effect which can be maximally obtained in a given biological system (Emax): therefore, the intrinsic activity can be defined as

$$α \sim \frac{{E}_{A}}{{E}_{ma x}}$$

The highest relative intrinsic activity results from E_{A}/Eₘₐₓ=1. Agonists with an intrinsic activity of 1 are full agonists, whereas substances/molecules with an intrinsic activity of >0 and <1 are partial agonists. Partial agonists show a dualistic effect, i.e. they comprise agonistic as well as antagonistic effects.

When used herein in connection with LRH-1 binding compounds, preferably LRH-1 agonists, the term “specific for LRH-1” means that a particular compound binds to LRH-1 to a statistically greater extent than to other biomolecules that may be present in a particular organism, e.g., at least 2, 3, 4, 5, 10, 20, 50, 100, or 1000-fold. Also, where biological activity other than binding is indicated, the term “specific for LRH-1” indicates that a particular compound has greater biological activity associated with binding to LRH-1 than to other biomolecules (e.g., at a level as indicated for binding specificity). Similarly, the specificity is preferably for LRH-1 and optionally for SF-1, but not for other nuclear receptors that may be present from an organism. In particular embodiments, the specificity is between SF-1 and LRH-1. Yet, particularly preferred the specificity is for LRH-1.

One approach to identifying agonists of LRH-1 is the ligand displacement assay, where the displacement of radiolabeled ligand by candidate agents is detected. Specifically, it is known from WO 2009/067182 that phosphatidylcholin (PC) lipids having 22-24 total carbon atoms in its fatty acid tails, each fatty acid tail is 10, 11, 12 or 13 carbon atoms in length such as diundecanoylphosphatidylcholine (DUPC), dilauroylphosphatidylcholine (DLPC), a C11:0, C12:0 undecanoyl, lauroyl PC, a C11:0, C13:0 undecanoyl, tridecanoyl PC, and a C10:0, C12:0 decanoyl, lauroyl PC or a combination thereof (in particular, DUPC or DLPC, or a combination thereof) act as agonists of LRH-1. Accordingly, one or more of these compounds is/are labelled with a detectable lable such as a radiolabel, fluorescent label and the like, contacted with LRH-1 (either present on cells or with recombinantly produced LRH-1 as described herein) and a candidate compounds. A potential agonist can be identified by displacement of the labelled compound from its ligand binding site on LRH-1. The more detectable label is released from LRH-1, the more ligand is displaced, the more is the likelihood that such a compound, which displaces a previously bound known LRH-1 ligand (agonist), could be a candidate for an LRH-1 agonist.

In the alternative to the above mentioned compounds, i.e., phosphatidylcholin (PC) lipids, a peptide derived from the coactivator TIF2 (amino acids 737-757) can be used.

Whether or not an LRH-1 ligand as identified above, might be an agonist can be, for example, tested as follows. A mammalian two-hybrid system can be used to test the interaction of a VP16-human LRH-1 ligand binding domain fusion with a second fusion of the Gal4 DNA binding domain to the receptor interaction domain of the LRH-1 coactivator SRC-3. If that interaction is stimulated by an LRH-1 ligand, the ligand is a candidate LRH-1 agonist. Likewise, if that interaction is unaffected by an LRH-1 ligand, the ligand might not necessarily qualify as an LRH-1 agonist. The results obtained with the two-hybrid system can, for example, optionally, be verified by a GST pulldown approach. Specifically, in the GST pulldown, the substantial basal interaction of E. coli expressed GST-LRH-1 with in vitro translated ³⁵S-labeled full length SRC-3 will be increased by a potential LRH-1 agonist. Otherwise, the substantial interaction will remain at its basal level. Quantitative measurement of the interaction in the GST pulldown can be done by imaging, for example, by using a PhosphorImager.

A functional test for an LRH-1 agonist may be as follows. It was previously established that LRH-1 regulates the expression of SHP within an autoregulatory feedback loop to control cholesterol metabolism in the liver (Goodwin et al. (2000), Mol. Cell 6:517-526). To determine as to whether an LRH-1 ligand might be an agonist, a heterologous reporter assay can be used. In said assay, mammalian cells are transfected with an expression vector for human LRH-1 and a reporter construct engineered from the proximal promoter of the human SHP gene fused to luciferase. Coexpression of the receptor and reporter gene will lead to an increase in luciferase due to the constitutive activity of LRH-1 in the absence of an exogenous ligand. However, if an LRH-1 ligand as identified by any of the methods described herein is added and will then further increase the reporter signal in a doseresponsive, is a candidate LRH-1 agonist.

Optionally, to further confirm the functional efficacy of a candidate LRH-1 agonist, human liver cells can be treated with the candidate LRH-1 agonist and SHP mRNA can be measured, preferably by quantitative PCR.

Suitable candidate LRH-1 agonists encompass numerous chemical classes, though typically they are organic compounds; preferably small organic compounds and are obtained from a wide variety of sources including libraries of synthetic or natural compounds. Another preferred class of LRH-1 agonists are of peptidic nature.

The crystal structure of LRH-1 was resolved (Krylova et al. (2005), Cell 120:343-355) and thus the crystal data can be used for the molecular design of LRH-1 ligands which are preferably agonists. Accordingly, the present invention also envisages a method of using the crystal data in a ligand screening assays, such as comprising: (a) selecting a potential ligand by performing structure assisted drug design with the three-dimensional structure determined for the crystal, wherein said selecting is performed in conjunction with computer modelling; optionally (b) contacting the potential ligand with the ligand binding domain of LRH-1 in an in vitro or in vivo assay, preferably with one or more described herein; and (c) detecting the binding of the potential ligand for the ligand binding domain.
The use of macromolecular crystallography as a tool for investigating ligand and receptor interactions, in particular structure-based drug design is reviewed in Oakley and Wilce (2000), Clin. Exp. Pharmacol. Physio. 27:145-151. The desired ligand is preferably an LRH-1 agonist that mimics endogenous transmitters or ligands.

Once the 3-D structure of the relevant target is known, computational processes can be used to search databases of compounds to identify ones that may interact strongly with the target. Lead compounds can be improved using the 3-D structure of the complex of the lead compound and its biological target. The activity of the selected compound can then be tested in a functional assay such as one of those described herein.

Preferably, the potential candidate LRH-1 agonist is selected on the basis of its having a greater affinity for the ligand binding domain of LRH-1 than that of a standard ligand for the ligand binding domain of LRH-1 such as the TIF-1 peptide described herein. However, though less preferred, the affinity of the selected compound may also be less than that of a standard ligand.

Such compounds are useful for example as a lead for the development of further analogues, which in turn may have enhanced binding affinity or otherwise beneficial therapeutic properties. On the other hand, the selected compound may bind to a site of the ligand-gated receptor other than known ligands.

Further the invention envisages a method further comprising: (d) forming a supplemental crystal of a protein-ligand complex by co-crystallization or soaking the crystal of LRH-1 with a potential ligand, wherein the crystal effectively diffracts X-rays for the determination of the atomic coordinates of the protein-ligand complex to a resolution of greater than 5.0, preferably greater than 4.0 Angstroms, more preferably greater than 3; (e) determining the three-dimensional structure of the supplemental crystal; (f) selecting a candidate ligand (preferably an agonist) by performing a structure assisted ligand design with the three-dimensional structure determined for the supplemental crystal, wherein said selecting is performed in conjunction with computer modelling; optionally (g) contacting the candidate ligand with a cell that expresses LRH-1; and (h) detecting a cell response; wherein a candidate ligand is identified as a drug when the cell response is altered, preferably when, for example, SHP mRNA expression will be increased as described herein compared to a cell that has not been contacted with the candidate ligand.

In view of the fact that LRH-1 was crystallized, the known LRH-1 crystal can also be used in X-ray crystallography-driven screening technique that combines the steps of lead identification, structural assessment, and optimization such as described for example in Nienaber et al., (2000), Nature Biotechnol. 18:1105-1108. This crystallographic screening method (named CrystaLEAD) has been used to sample large compound libraries and detecting ligands by monitoring changes in the electron density map of the crystal relative to the unbound form. The electron density map yields a high-resolution picture of the ligand-protein complex that provides key information to a structure-directed drug discovery process. The bound ligand is directly visualized in the electron density map. Ligands that bind away from the targeted site may be eliminated. The above described methods can be coupled with state-of-the-art laboratory data collection facilities including CCD detectors and data acquisition robotics.

Another method for LRH-1 ligand, preferably LRH-1 agonist design that could be applied in the invention comprises the step of using the structural coordinates of the LRH-1 protein crystal comprising the coordinates of Table 3 of US 2006/0160135, to computationally evaluate a chemical entity for associating with the ligand-binding site or a non-specific binding site of LRH-1. This approach, made possible and enabled by this invention, is to screen computationally small molecule data bases for chemical entities or compounds that can bind in whole, or in part, to LRH-1. In this screening, the quality of fit of such entities or compounds to the binding site may be judged either by shape complementarity or by estimated interaction energy (Meng et al. (1992), J. Comp. Chem. 13:505-524).

Preferred coordinates of a LRH-1 crystal are from Table 3 of US 2006/0160135 (incorporated by reference).

Preferred crystallographic data and refinement of a LRH-1 crystal that can be applied in identifying a LRH-1 agonist are from Wang et al. (2005), PNAS 102:7505-7510:

| | hSF-1 | hLRH-1 |
|---|---|---|
| Crystal and data collection statistics | | |
| Unit cell dimensions, Å | a = b = 73.6 | a = 61.0, b = |
| | c = 195.7 | 67.0 c = 78.2 |
| Space group | P3₁2₁ | P2₁2₁2₁ |
| Solvent content | 49% | 53% |
| Resolution range, Å | 50-2.1 | 50-2.5 |
| Unique reflections | 36,333 | 10,899 |
| Data redundancy | 4.2 | 4.6 |
| Completeness, % | 98.7 | 99.4 |
| <I/σ(/)> | 6.9 | 10.0 |
| R_{sym}, % | 11.2 | 4.9 |
| Refinement statistics | | |
| or cut off | None | None |
| Total non-hydrogen atoms | 4,342 | 2,172 |
| Ave B factor, Å² | 24.0 | 34.2 |
| R_{cryst}/R_{free}, % | 21.6/26.5 | 23.9/28.1 |
| rms deviation bond lengths, Å | 0.012 | 0.008 |
| rms deviation bond angles, * | 1.449 | 1.034 |

R_{sym} = Σ|f_{avg} − Iⱼ/ΣIⱼ. R_{cryst} = Σ|Fₒ − F_{c}|/ΣFₒ, where Fₒ and F_{c} are the observed and calculated structure factors, respectively, R_{free} was calculated from a randomly chosen 5% of reflections excluded from the refinement, and R_{cryst} was calculated from the remaining 95% of reflections, rms deviation values are from ideal geometry.

or from Krylova et al. (2005), Cell 120:343-355:

| | | | | |
|---|---|---|---|---|
| | | hSF-1/ | hSF-1/ | hLRH-1/ |
| Crystalizations | mSF-1/SHP-1 | No Pep. | TIF-2 | TIF-2 |

Unit Cell Dimensions

| | | | | |
|---|---|---|---|---|
| a ( Å) | 73.9 | 128.4 | 73.1 | 69.9 |
| b (Å) | 79.9 | 66.0 | 73.1 | 67.2 |
| c (Å) | 117.0 | 141.0 | 139.4 | 79.6 |
| Space group | P4₁2₁2 | P2₁ | P4₁2₁2 | P2₁2₁2 |
| Molecules per asymmetric unit | 1 | 7 | 1 | 1 |
| Resolution (Å) | 25-1.2 | 20-2.5 | 20-2.9 | 40-2.5 |
| Number of unique | 100,491 | 71,693 | 15,614 | 10,787 |
| reflections | | | | |
| Data redundancy | 13.1 | 2 | 8 | 6 |
| Completeness (%) | 99.9 | 97.9 | 99.8 | 91.9 |
| R_{symm} (%) | 6.1 | 4.5 | 5.8 | 3.7 |
| <I/o(l)> | 39.2 | 28.0 | 30.0 | 44.0 |

Refinement

| | | | | |
|---|---|---|---|---|
| R | 19.7 | 24.9 | 24.6 | 22.2 |
| Rfree | 21.1 | 28.3 | 29.0 | 28.3 |

Rms deviation from identity

| | | | | |
|---|---|---|---|---|
| Bond length (Å) | 0.012 | 0.009 | 0.011 | 0.008 |
| Bond angle (0) | 1.483 | 1.20 | 1.34 | 1.349 |

Average B factor (Å2)

| | | | | |
|---|---|---|---|---|
| All atoms | 22.8 | 85 | 62 | 64.4 |
| Protein atoms | 21.3 | 85 | 61 | 63.9 |
| Water molecules | 25.7 | 78 | 55 | 60.5 |

In addition, in accordance with this invention, LRH-1 mutants or chimerics may be crystallized in co-complex with known ligands such as phosphatidylcholins or BL 001 as described herein. The crystal structures of a series of such complexes may then be solved by molecular replacement (for review see for example Brunger et al. (1999), Prog. Biophys. Mol. Biol. 72:135-155; and references cited therein) and compared with that of wild-type LRH-1. Potential sites for modification within the various binding sites of the ligand-binding domain may thus be identified. This information provides an additional tool for determining the most efficient binding interactions, for example, increased hydrophobic interactions, between LRH-1 and a chemical entity or compound.

The design of compounds that bind to LRH-1, thereby preferably acting as agonist according to this invention generally involves consideration of two factors.

First, the compound must be capable of physically and structurally associating with the ligand-binding domain. Non-covalent molecular interactions important in the association of the ligand-binding domain with its ligand include hydrogen bonding, van der Waals and hydrophobic interactions.

Second, the compound must be able to assume a conformation that allows it to associate with the ligand-binding domain. Although certain portions of the compound will not directly participate in this association, those portions may still influence the overall conformation of the molecule. This, in turn, may have a significant impact on potency. Such conformational requirements include the overall three-dimensional structure and orientation of the chemical entity or compound in relation to all or a portion of the binding site or the spacing between functional groups of a compound comprising several chemical entities that directly interact with the LRH-1.

If the theoretical structure of the given compound suggests insufficient interaction and association between it and LRH-1, synthesis and testing of the compound is obviated. However, if computer modelling indicates a strong interaction, the molecule may then be synthesized and tested for its ability to bind to LRH-1 and functionally tested according to the methods mentioned above. In this manner, synthesis of inoperative compounds may be avoided. Once suitable chemical entities or fragments have been selected, they can be assembled into a single compound or inhibitor. Assembly may be proceed by visual inspection of the relationship of the fragments to each other on the three-dimensional image displayed on a computer screen in relation to the structure coordinates of LRH-1. This would be followed by manual model building using software such as Quanta or Sybyl.

Useful programs to aid one of skill in the art in connecting the individual chemical entities or fragments include CAVEAT (Bartlett, et al. (1989), “CAVEAT: A Program to Facilitate the Structure-Derived Design of Biologically Active Molecules”. In Molecular Recognition in Chemical and Biological Problems”, Special Pub., Royal Chem. Soc. 78:182-196); 3D Database systems such as MACCS-3D (Martin (1992), J. Med. Chem. 35:2145-2154) and HOOK (Molecular Simulations, Burlington, Mass.).

Instead of proceeding to build an LRH-1 agonist in a step-wise fashion one fragment or chemical entity at a time as described above, LRH-1 agonist compounds may be designed as a whole or “de novo” using either an empty active site or optionally including some portion (s) of a known ligand (s). These methods include LUDI (Bohm, J. Comp. Aided. Mol. Design. 6 (1992), 61-78); LEGEND (Nishibata and Itai, Tetrahedron 47 (1991), 8985); and LeapFrog (Tripos Associates, St. Louis, Mo.).

Other molecular modeling techniques may also be employed in accordance with this invention; see, e.g., Cohen (1990), J. Med. Chem. 33:883-894 and Navia and Murcko (1992), Current Opinions in Structural Biology 2: 202-210. Such computer modeling is preferably performed with a Docking program (Dunbrack et al. (1997), Protein Sci. 6:1661-1681 and Folding Des. 2 (1997), R27-R42).

Methods for the identification of drugs or corresponding lead compounds in computational prescreen using X-ray crystal structures are described in the prior art (Verlinde and Hol (1994), Structure 2:577-587; Kuntz (1992), Science 257:1078-1082; Shuker et al. (1996), Science 274:1531-1534; Fejzo et al. (1999), Chem. Biol. 6:755-769; WO 98/58961). The structural information can be consulted to efficiently optimize leads. Computational programs have been written to identify compounds ranging from very small molecules or functional groups (GRID: Goodford, J. (1985), Med. Chem. 28:849-857; MCSS: Caflish et al. (1993), J. Med. Chem. 36:2142-2167) to potential lead scaffolds (DOCK: Kuntz et al. (1994), Accounts Chem. Res. 27:117-123) using solved X-ray crystal structures. Another method computationally prescreens compound libraries and experimentally tests the individual “hits” by X-ray crystallography (Verlinde et al. (1992), J. Comput. Aided Mol. Des. 6:131-147) in order to decrease the size of the screening library. In addition, an experimental approach has been developed to find organic solvents that bind to active sites that may be recombined into a lead macromolecule (Allen et al. (1996), J. Phys. Chem. 100: 2605-2611).

Once a compound has been designed or selected by the above methods, the efficiency with which that compound may bind to LRH-1, preferably its ligand-binding domain, may be tested and optimized by computational evaluation.

A compound designed or selected as binding to LRH-1 may be further computationally optimized so that in its bound state it would preferably lack repulsive electrostatic interaction with the target ligand-binding domain. Such non-complementary (e.g., electrostatic) interactions include repulsive charge-charge, dipole-dipole and charge-dipole interactions. Specifically, the sum of all electrostatic interactions between the ligand and LRH-1 when the ligand is bound to LRH-1, preferably make a neutral or favorable contribution to the enthalpy of binding.

Specific computer software is available in the art to evaluate compound deformation energy and electrostatic interaction. Examples of programs designed for such uses include Gaussian 92, revision C (Frisch, Gaussian, Inc., Pittsburgh, Pa.); AMBER, version 4.0 (Kollman, University of California at San Francisco); QUANTA/CHARMM (Molecular Simulations, Inc., Burlington, Mass.); and Insight II/Discover (Biosysm Technologies Inc., San Diego, Calif.). These programs may be implemented, for instance, using a Silicon Graphics workstation, IRIS 4D/35, IBM RISC/6000 workstation model 550 or better a Unix workstation (SGI, Alpha, Sun, etc.) or any Linux PC. Other hardware systems and software packages will be known to those skilled in the art.

Once an LRH-1 ligand, preferably an LRH-1 agonist has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to LRH-1 by the same computer methods described in detail, above.

As mentioned before, the above described methods of the present invention can also be used as an initial drug screening assay followed by a classical drug screening assay using the biochemical assays known in the art.

The design and preparation of LRH-1 agonists can be performed with or without structural and/or co-crystallization data by considering the chemical structures in common between the active scaffolds of a set.

By “molecular scaffold” or “scaffold” is meant a small target binding molecule to which one or more additional chemical moieties can be covalently attached, modified, or eliminated to form a plurality of molecules with common structural elements. The moieties can include, but are not limited to, a halogen atom, a hydroxyl group, a methyl group, a nitro group, a carboxyl group, or any other type of molecular group including, but not limited to, those recited in this application. Molecular scaffolds bind to at least one target molecule with low or very low affinity and/or bind to a plurality of molecules in a target family (e.g., protein family), and the target molecule is preferably an enzyme, receptor, or other protein. Preferred characteristics of a scaffold include molecular weight of less than about 350 daltons; binding at a target molecule binding site such that one or more substituents on the scaffold are situated in binding pockets in the target molecule binding site; having chemically tractable structures that can be chemically modified, particularly by synthetic reactions, so that a combinatorial library can be easily constructed; having chemical positions where moieties can be attached that do not interfere with binding of the scaffold to a protein binding site, such that the scaffold or library members can be modified to form ligands, to achieve additional desirable characteristics, e.g., enabling the ligand to be actively transported into cells and/or to specific organs, or enabling the ligand to be attached to a chromatography column for additional analysis. Thus, a molecular scaffold is a small, identified target binding molecule prior to modification to improve binding affinity and/or specificity, or other pharmacologic properties.

In this design process structure-activity hypotheses can be formed and those chemical structures found to be present in a substantial number of the scaffolds, including those that bind with low affinity, can be presumed to have some effect on the binding of the scaffold. This binding can be presumed to induce a desired biochemical effect when it occurs in a biological system (e.g., a treated mammal). New or modified scaffolds or combinatorial libraries derived from scaffolds can be tested to disprove the maximum number of binding and/or structure-activity hypotheses. The remaining hypotheses can then be used to design ligands that achieve a desired binding and biochemical effect.

But in many cases it will be preferred to have co-crystallography data for consideration of how to modify the scaffold to achieve the desired binding effect (e.g., binding at higher affinity or with higher selectivity). Using the case of proteins and enzymes, co-crystallography data shows the binding pocket of the protein with the molecular scaffold bound to the binding site, and it will be apparent that a modification can be made to a chemically tractable group on the scaffold. For example, a small volume of space at a protein binding site or pocket might be filled by modifying the scaffold to include a small chemical group that fills the volume. Filling the void volume can be expected to result in a greater binding affinity, or the loss of undesirable binding to another member of the protein family. Similarly, the co-crystallography data may show that deletion of a chemical group on the scaffold may decrease a hindrance to binding and result in greater binding affinity or specificity.

Various software packages have implemented techniques which facilitate the identification and characterization of interactions of potential binding sites from complex structure, or from an apo structure of a target molecule, i.e. one without a compound bound (e.g. SiteID, Tripos Associates, St. Louis Mo. and SiteFinder, Chemical Computing Group, Montreal Canada, GRID, Molecular Discovery Ltd., London UK). Such techniques can be used with the coordinates of a complex between the scaffold of interest and a target molecule, or these data in conjunction with data for a suitably aligned or superimposed related target molecule, in order to evaluate changes to the scaffold that would enhance binding to the desired target molecule structure or structures. Molecular Interaction Field-computing techniques, such as those implemented in the program GRID, result in energy data for particular positive and negative binding interactions of different computational chemical probes being mapped to the vertices of a matrix in the coordinate space of the target molecule. These data can then be analyzed for areas of substitution around the scaffold binding site which are predicted to have a favourable interaction for a particular target molecule. Compatible chemical substitution on the scaffold e.g. a methyl, ethyl or phenyl group in a favourable interaction region computed from a hydrophobic probe, would be expected to result in an improvement in affinity of the scaffold. Conversely, a scaffold could be made more selective for a particular target molecule by making such a substitution in a region predicted to have an unfavourable hydrophobic interaction in a second, related undesirable target molecule.

It can be desirable to take advantage of the presence of a charged chemical group located at the binding site or pocket of the protein. For example, a positively charged group can be complemented with a negatively charged group introduced on the molecular scaffold. This can be expected to increase binding affinity or binding specificity, thereby resulting in a more desirable ligand. In many cases, regions of protein binding sites or pockets are known to vary from one family member to another based on the amino acid differences in those regions. Chemical additions in such regions can result in the creation or elimination of certain interactions (e.g., hydrophobic, electrostatic, or entropic) that allow a compound to be more specific for one protein target over another or to bind with greater affinity, thereby enabling one to synthesize a compound with greater selectivity or affinity for a particular family member. Additionally, certain regions can contain amino acids that are known to be more flexible than others. This often occurs in amino acids contained in loops connecting elements of the secondary structure of the protein, such as alpha helices or beta strands. Additions of chemical moieties can also be directed to these flexible regions in order to increase the likelihood of a specific interaction occurring between the protein target of interest and the compound. Virtual screening methods can also be conducted in silico to assess the effect of chemical additions, subtractions, modifications, and/or substitutions on compounds with respect to members of a protein family or class.

The addition, subtraction, or modification of a chemical structure or sub-structure to a scaffold can be performed with any suitable chemical moiety. For example the following moieties, which are provided by way of example and are not intended to be limiting, can be utilized: hydrogen, alkyl, alkoxy, phenoxy, alkenyl, alkynyl, phenylalkyl, hydroxyalkyl, haloalkyl, aryl, arylalkyl, alkyloxy, alkylthio, alkenylthio, phenyl, phenylalkyl, phenylalkylthio, hydroxyalkyl-thio, alkylthiocarbbamylthio, cyclohexyl, pyridyl, piperidinyl, alkylamino, amino, nitro, mercapto, cyano, hydroxyl, a halogen atom, halomethyl, an oxygen atom (e.g., forming a ketone or N-oxide) or a sulphur atom (e.g., forming a thiol, thione, di-alkylsulfoxide or sulfone) are all examples of moieties that can be utilized.

Additional examples of structures or sub-structures that may be utilized are an aryl optionally substituted with one, two, or three substituents independently selected from the group consisting of alkyl, alkoxy, halogen, trihalomethyl, carboxylate, nitro, and ester moieties; an amine of formula —NX₂X₃, where X₂ and X₃ are independently selected from the group consisting of hydrogen, saturated or unsaturated alkyl, and homocyclic or heterocyclic ring moieties; halogen or trihalomethyl; a ketone of formula —COX₄, where X₄ is selected from the group consisting of alkyl and homocyclic or heterocyclic ring moieties; a carboxylic acid of formula —(X₅)ₙCOOH or ester of formula (X₆)ₙCOOX₇, where X₅, X₆, and X₇ and are independently selected from the group consisting of alkyl and homocyclic or heterocyclic ring moieties and where n is 0 or 1; an alcohol of formula (X₈)ₙOH or an alkoxy moiety of formula —(X₈)ₙOX₉, where X₈ and X₉ are independently selected from the group consisting of saturated or unsaturated alkyl and homocyclic or heterocyclic ring moieties, wherein said ring is optionally substituted with one or more substituents independently selected from the group consisting of alkyl, alkoxy, halogen, trihalomethyl, carboxylate, nitro, and ester and where n is 0 or 1; an amide of formula NHCOX₁₀, where X₁₀ is selected from the group consisting of alkyl, hydroxyl, and homocyclic or heterocyclic ring moieties, wherein said ring is optionally substituted with one or more substituents independently selected from the group consisting of alkyl, alkoxy, halogen, trihalomethyl, carboxylate, nitro, and ester; SO₂, NX₁₁, X₁₂, where X₁₁ and X₁₂ are selected from the group consisting of hydrogen, alkyl, and homocyclic or heterocyclic ring moieties; a homocyclic or heterocyclic ring moiety optionally substituted with one, two, or three substituents independently selected from the group consisting of alkyl, alkoxy, halogen, trihalomethyl, carboxylate, nitro, and ester moieties; an aldehyde of formula —COH; a sulfone of formula —SO₂X₁₃, where X₁₃ is selected from the group consisting of saturated or unsaturated alkyl and homocyclic or heterocyclic ring moieties; and a nitro of formula —NO₂.

It can also be beneficial in selecting compounds for testing to first identify binding characteristics that a ligand should advantageously possess. This can be accomplished by analyzing the interactions that a plurality of different binding compounds have with a particular target, e.g., interactions with one or more conserved residues in the binding site. These interactions are identified by considering the nature of the interacting moieties. In this way, atoms or groups that can participate in hydrogen bonding, polar interactions, charge-charge interactions, and the like are identified based on known structural and electronic factors.

In addition to the identification and development of ligands, determination of the orientation of a molecular scaffold or other binding compound in a binding site allows identification of energetically allowed sites for attachment of the binding molecule to another component. For such sites, any free energy change associated with the presence of the attached component should not destabilize the binding of the compound to the target to an extent that will disrupt the binding. Preferably, the binding energy with the attachment should be at least 4 kcal/mol., more preferably at least 6, 8, 10, 12, 15, or 20 kcal/mol. Preferably, the presence of the attachment at the particular site reduces binding energy by no more than 3, 4, 5, 8, 10, 12, or 15 kcal/mol.

In many cases, suitable attachment sites will be those that are exposed to solvent when the binding compound is bound in the binding site. In some cases, attachment sites can be used that will result in small displacements of a portion of the enzyme without an excessive energetic cost. Exposed sites can be identified in various ways. For example, exposed sites can be identified using a graphic display or 3-dimensional model. In a graphic display, such as a computer display, an image of a compound bound in a binding site can be visually inspected to reveal atoms or groups on the compound that are exposed to solvent and oriented such that attachment at such atom or group would not preclude binding of the enzyme and binding compound. Energetic costs of attachment can be calculated based on changes or distortions that would be caused by the attachment as well as entropic changes.

Many different types of components can be attached. Persons with skill are familiar with the chemistries used for various attachments. Examples of components that can be attached include, without limitation: solid phase components such as beads, plates, chips, and wells; a direct or indirect label; a linker, which may be a traceless linker; among others. Such linkers can themselves be attached to other components, e.g., to solid phase media, labels, and/or binding moieties.

The binding energy of a compound and the effects on binding energy for attaching the molecule to another component can be calculated approximately by manual calculation, or by using any of a variety of available computational virtual assay techniques, such as docking or molecular dynamics simulations. A virtual library of compounds derived from the attachment of components to a particular scaffold can be assembled using a variety of software programs (such as Afferent, MDL Information Systems, San Leandro, Calif. or CombiLibMaker, Tripos Associates, St. Louis, Mo.). This virtual library can be assigned appropriate three dimensional coordinates using software programs (such as Concord, Tripos Associates, St. Louis, Mo. or Omega, Openeye Scientific Software, Santa Fe, N.Mex.). These structures may then be submitted to the appropriate computational technique for evaluation of binding energy to a particular target molecule. This information can be used for purposes of prioritizing compounds for synthesis, for selecting a subset of chemically tractable compounds for synthesis, and for providing data to correlate with the experimentally determined binding energies for the synthesized compounds.

The crystallographic determination of the orientation of the scaffold in the binding site specifically enables more productive methods of assessing the likelihood of the attachment of a particular component resulting in an improvement in binding energy. Such an example is shown for a docking-based strategy in Hague et al. (J. Med. Chem. (1999), 42:1428-1440), wherein an “Anchor and Grow” technique which relied on a crystallographically determined fragment of a larger molecule, potent and selective inhibitors were rapidly created. The use of a crystallographically characterized small molecule fragment in guiding the selection of productive compounds for synthesis has also been demonstrated in Boehm et al., (J. Med. Chem. (2000), 43:2664-2674). An illustration of the use of crystallographic data and molecular dynamics simulations in the prospective assessment of inhibitor binding energies can be found in Pearlman and Charifson (J. Med. Chem. (2001), 44, 3417-3423). Another important class of techniques which rely on a well defined structural starting point for computational design is the combinatorial growth algorithm based systems, such as the GrowMol program (Bohacek & McMartin, J. Amer. Chem. Soc. (1994), 116:5560-71. These techniques have been used to enable the rapid computational evolution of virtual inhibitor computed binding energies, and directly led to more potent synthesized compounds whose binding mode was validated crystallographically (see Organic Letters, 2001, 3:2309-2312).

The LRH-1 agonist applied in the embodiments of the invention preferably interacts with one or more, more preferably with all, of the residues given in the column “hSF-1” and/or “hLRH-1”, more preferably with all of these residues. More preferably, the LRH-1 agonist preferably interacts with one or more, more preferably with all. of the residues given in the column “hLRH-1”.

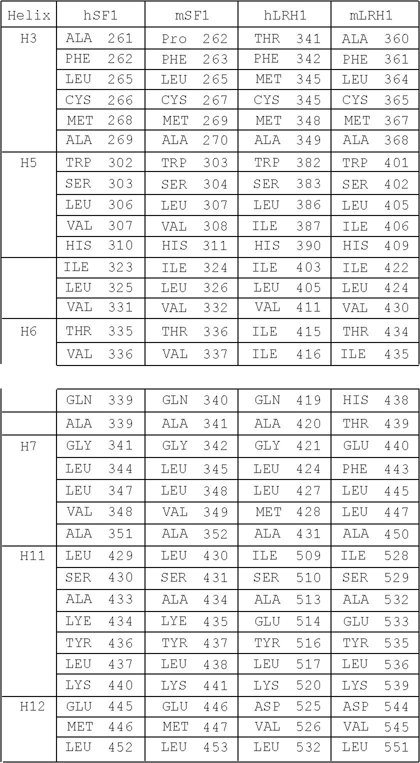

Residues contacting the ligand are shown for all four receptors; all distances are less than 5 Å. Intra-receptor differences are highlighted in dark grey, while inter-receptor differences are shown in light grey. The corresponding positions of each residue relative to helices are indicated in the first column, regions spanning helices are left blank.

The “reference sequence” for hLRH-1 according to which the numbering of the amino acid residues is done is the hLRH-1 amino acid sequence as deposited with GenBank under Accession No. AAG17125.

The “reference sequence” for hSF-1 according to which the numbering of the amino acid residues is done is the hSF-1 amino acid sequence as deposited with GenBank under Accession No. AAB53105.

The skilled person knows that any hLRH-1 or hSF-1 amino acid sequence can be brought into relation with the reference sequence for hLRH-1 as deposited with GenBank under Accession No. AAG17125 or for hSF-1 as deposited with Gen Bank under Accession No. AAB53105, by way of an alignment. In particular, a residue at a certain position in the reference sequence may not necessarily be located at the very same position in a sequence which is aligned with the reference sequence. This is so because the reference sequence is somewhat shorter or longer. Accordingly, a certain residue of the reference sequence thus “corresponds” to a certain residue in a sequence aligned with the reference sequence. The same is true when nucleotide sequences are aligned.

Therefore, the term “corresponding” as used herein means that a position is not only determined by the number of the preceding nucleotides and amino acids, respectively. Rather, the position of a given nucleotide or amino acid may vary in comparison to the position of a sequence aligned with the given nucleotide or amino acid sequence due to deletions or additions. Thus, under a “corresponding position” in accordance with the present invention it is to be understood that nucleotides or amino acids may differ in the indicated number but may still have similar neighbouring nucleotides or amino acids. Said nucleotides or amino acids which may be exchanged, deleted or comprise additional nucleotides or amino acids are also comprised by the term “corresponding position”.

Even more preferably The LRH-1 agonist applied in the embodiments of the invention even more preferably interacts with a Lys from H11 (K440/hSF-1 or K520/hLRH-1), a Tyr from H11 (Y436/hSF-1 or Y516/hLRH-1) and a Gly from the H6-H7 loop (G341/hSF-1 or G421/hLRH-1). Amino acid positions are numbered in relation to the hSF-1 reference sequence as deposited with GenBank Accession No. AAB53105 and in relation to the hLRH-1 reference sequence as deposited with GenBank Accession No. AAG17125.

When the sequence as deposited with GenBank under Accession No. NM_{—}004959 is used as reference sequence for hSF-1, the numbering remains unchanged, since the sequence deposited under NM_{—}004959 and AAB53105 are identical.

However, when sequence as deposited with GenBank under Accession No. NM_{—}003822 is used as reference sequence for hLRH-1, then amino acid residue at K520 of LRH-1, Y516 of LRH-1 and G421 of LRH-1 of the reference sequence as deposited with GenBank under Accession No. AAB53105 corresponds to position K474 of LRH-1, Y470 of LRH-1 and G375 of LRH-1 (see also Wang et all. (2005), PNAS 102:7505-7510 who uses NM_{—}003822).

In another preferred embodiment the LRH-1 agonist applied in the uses and methods of the invention is a compound having formula (Ia)

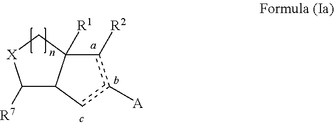

wherein
A is methyl, ethyl, aryl (preferably phenyl), cycloalkyl (preferably cyclohexyl) or

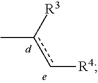

X is C(R)₂ or NR; preferably X is CH₂;
n is 1 or 2; preferably n is 1;
R is H, alkyl (preferably methyl) or R is OR¹⁰ wherein R¹⁰ is H, alkyl, acyl;
R¹ is —N(R⁵)₂, OR¹¹ or C(R¹²)═CH₂;
R² is H, alkyl, halogen, alkenyl, alkynyl, aryl, aralkyl, aralkenyl, alkylene, alkenylene, cycloalkyl, cycloalkylene or N-heterocyclyl which can be optionally substituted;
R³ and R⁴ are independently H, alkyl, alkenyl, alkynyl, aryl, aralkyl, aralkenyl, alkylene, alkenylene, cycloalkyl, cycloalkylene, halogen or N-heterocyclyl;
each R⁵ is independently hydrogen, alkyl, alkenyl, alkynyl, aryl, aralkyl or aralkenyl;
R⁷ is H, OH, OR⁸ wherein R⁸ is alkyl, acyl or aryl; and
R¹¹ is C₂-C₄ optionally substituted C₂-C₄ alkyl, optionally substituted aralkyl (preferably benzyl), optionally substituted cycloalkyl (preferably cyclohexyl); R¹¹ is C₂-C₄ optionally substituted alkyl, (it seem that with some alkyl is not so good (page 2272) and table 7. Hence I limit to C₂-C₄
R¹² is optionally substituted aryl (preferably optionally substituted phenyl, preferably the substituent on the phenyl is selected from para-methyl, para-buthyl, meta-methoxy, or R¹² is C₂-C₄ alkyl;
wherein when A is

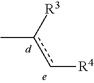

there is independently a maximum of one double bond between each of the carbon atoms of the centers a-b and b-c and d-e; and
when A is methyl, aryl (preferably phenyl) or cycloalkyl (preferably cyclohexyl) there is independently a maximum of one double bond between each of the carbon atoms of the centers a-b and b-c.

Preferably in formula (Ia):
X is C(R)₂ or NR; preferably X is CH₂;
n is 1 or 2; preferably n is 1;
R is H, alkyl (preferably methyl), aralkyl (preferably benzyl), OR¹⁰ wherein R¹⁰ is H, alkyl, acyl; and
   when R¹ is —N(R⁵)₂, or OR¹¹
R² is H, alkyl, alkoxyl, halogen, alkenyl, alkynyl, aryl, aralkyl, aralkenyl, alkylene, alkenylene, cycloalkyl, cycloalkylene or N-heterocyclyl which can be optionally substituted
R³ and R⁴ are independently H, alkyl, alkenyl, alkynyl, aryl, aralkyl, aralkenyl, alkylene, alkenylene, cycloalkyl, cycloalkylene, halogen or N-heterocyclyl;
each R⁵ is independently hydrogen, alkyl, alkenyl, alkynyl, aryl, aralkyl or aralkenyl;
R⁷ is H, OH, OR⁸ wherein R⁸ is alkyl, acyl or aryl; and
R¹¹ is C₂-C₄ optionally substituted alkyl, optionally substituted aralkyl (preferably benzyl), optionally substituted cycloalkyl (preferably cyclohexyl); and
   when R¹ is C(R¹²)═CH₂
n is 1;
R¹² is optionally substituted aryl (preferably optionally substituted phenyl; preferably the substituent on the phenyl is in position meta) or R¹² is C₂-C₄ alkyl;
R² is alkoxy (preferably C₁-C₃ alkoxy, more preferably propyloxy), halogen, alkenyl, alkynyl, aryl, aralkyl, aralkenyl, alkylene, alkenylene, cycloalkyl, cycloalkylene or N-heterocyclyl which can be optionally substituted, preferably R² is alkyoxy, preferably C₁-C₃ alkoxyl, more preferably propyloxy or aryl, preferably phenyl;
R³ and R⁴ are independently H, alkyl, alkenyl, alkynyl, aryl, aralkyl, aralkenyl, alkylene, alkenylene, cycloalkyl, cycloalkylene, halogen or N-heterocyclyl;
each R⁵ is independently hydrogen, alkyl, alkenyl, alkynyl, aryl, aralkyl or aralkenyl;
R⁷ is H, OH, OR⁸ wherein R⁸ is alkyl, acyl or aryl, preferably R⁷ is “endo”;
and
wherein when A is

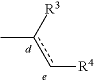

there is independently a maximum of one double bond between each of the carbon atoms of the centers a-b and b-c and d-e, preferably when R1 is C(R¹²)═CH₂ the bond between carbon atom of centers a-b is a double bond
and when A is methyl, aryl (preferably phenyl) or cycloalkyl (preferably cyclohexyl) there is independently a maximum of one double bond between each of the carbon atoms of the centers a-b and b-c.
Preferably when R¹² is phenyl, R² is aryl, preferably phenyl or hydroxy-substituted alkyl, preferably hydroxyl-propyl, A is alkyl, preferably n-hexyl and R⁷ is H or OH, n is 1 and X is CH₂.

In another preferred embodiment the LRH-1 agonist applied in the uses and methods of the invention is a 9-substituted bicycle [3.3.0] octane derivative, including a substituted cis-bicyclo[3.3.0]-oct-2-ene derivative, of the general formula I:

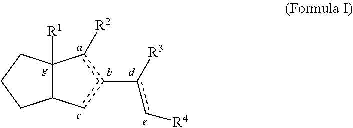

The compound shown in formula I is generally classed as 9-substituted bicyclo [3.3.0] octane derivative (wherein the 9-position pertains to the prostaglandin numbering system).

In the context of the 9-substituted bicyclo [3.3.0] octane derivative the following terms have the meaning indicated:

### R¹ is —N(R⁵)₂

R² is H, alkyl, halogen, alkenyl, alkynyl, aryl, aralkyl, aralkenyl, alkylene, alkenylene, cycloalkyl, cycloalkylene or N-heterocyclyl (which can be optionally substituted as described herein below).
There is independently a maximum of one double bond between each of the carbon atoms of the centers a-b and b-c and d-e.
R³ and R⁴ are independently alkyl, alkenyl, alkynyl, aryl, aralkyl, aralkenyl, alkylene, alkenylene, cycloalkyl, cycloalkylene, halogen or N-heterocyclyl.
Each R⁵ is independently hydrogen, alkyl, alkenyl, alkynyl, aryl, aralkyl or aralkenyl.
Each R⁶ is a straight or branched alkylene chain optionally substituted by hydroxy, mercapto, alkylthio, aryl, cycloalkyl, —N(R⁵)₂, —C(O)OR⁵ or —C(O)N(R⁵)₂.

“Alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like. Unless stated otherwise specifically in the specification, the alkyl radical may be optionally substituted by hydroxy, alkoxy, aryloxy, haloalkoxy, cyano, nitro, mercapto, alkylthio, cycloalkyl, —N(R⁵)₂—C(O)OR⁵, —C(O)N(R⁵)₂ or —N(R⁵)—C(O)—R⁵ where each R⁵ is as defined above.

Unless stated otherwise specifically in the specification, it is understood that for radicals, as defined below, that contain a substituted alkyl group that the substitution can occur on any carbon of the alkyl group.

“Alkoxy” refers to a radical of the formula —ORₐ where Rₐ is an alkyl radical as defined above, e.g., methoxy, ethoxy, n-propoxy, 1-methylethoxy (iso-propoxy), n-butoxy, n-pentoxy, 1,1-dimethylethoxy (t-butoxy), and the like. Unless stated otherwise specifically in the specification, it is understood that for radicals, as defined below, that contain a substituted alkoxy group that the substitution can occur on any carbon of the alkoxy group. The alkyl radical in the alkoxy radical may be optionally substituted as described above.

“Alkylthio” refers to a radical of the formula —SRₐ where Rₐ is an alkyl radical as defined above, e.g., methylthio, ethylthio, n-propylthio, 1-methylethylthio (iso-propylthio), n-butylthio, n-pentylthio, 1,1-dimethylethylthio (t-butylthio), and the like. Unless stated otherwise specifically in the specification, it is understood that for radicals, as defined below, that contain a substituted alkylthio group that the substitution can occur on any carbon of the alkylthio group. The alkyl radical in the alkylthio radical may be optionally substituted as described above.

“Alkenyl” refers to a straight or branched hydrocarbon chain radical consisting solely of carbon and hydrogen atoms, containing at least one double bond, having from two to eight carbon atoms, and which is attached to the rest of the molecule by a single bond or a double bond, e.g., ethenyl, prop-1-enyl, but-1-enyl, pent-1-enyl, penta-1,4-dienyl, and the like. Unless stated otherwise specifically in the specification, the alkenyl radical may be optionally substituted by hydroxy, alkoxy, haloalkoxy, cyano, nitro, mercapto, alkylthio, cycloalkyl, —N(R⁵)₂, —C(O)OR⁵, —C(O)N(R⁵)₂ or —N(R⁵)—C(O)—R⁵ where each R⁵ is as defined above. Unless stated otherwise specifically in the specification, it is understood that for radicals, as defined below, that contain a substituted alkenyl group that the substitution can occur on any carbon of the alkenyl group.

“Alkynyl” refers to a straight or branched monovalent hydrocarbon chain radical consisting solely of carbon and hydrogen atoms, containing at least one triple bond, having from two to eight carbon atoms, and which is attached to the rest of the molecule by a single bond, e.g., ethynyl, prop-1-ynyl, but-1-ynyl, pent-1-ynyl, pent-3-ynyl, and the like. Unless stated otherwise specifically in the specification, the alkynyl radical may be optionally substituted by hydroxy, alkoxy, haloalkoxy, cyano, nitro, mercapto, alkylthio, cycloalkyl, —N(R⁵)₂, —C(O)OR⁵, —C(O)N(R⁵)₂ or —N(R⁵)—C(O)—R⁵ where each R⁵ is as defined above. Unless stated otherwise specifically in the specification, it is understood that for radicals, as defined below, that contain a substituted alkynyl group that the substitution can occur on any carbon of the alkynyl group.

“Aryl” refers to a phenyl or naphthyl radical. Unless stated otherwise specifically in the specification, the term “aryl” or the prefix “ar-” (such as in “aralkyl”) is meant to include aryl radicals optionally substituted by one or more substituents selected from the group consisting of alkyl, halo, nitro, cyano, haloalkyl, haloalkoxy, mercapto, alkylthio, phenyl, cycloalkyl, —OR⁵ (including hydroxy and alkoxy), —N(R⁵)₂, —R⁶—N(R⁵)₂, —N(R⁵)—C(O)OR⁵, —R⁶—N(R⁵)—C(O)OR⁵, —N(R⁵)—C(O)—R⁵, —R⁶—N(R⁵)—C(O)—R⁵, —C(O)OR⁵, —R⁶—C(O)OR⁵, —C(O)—N(R⁵)₂, —R⁶—C(O)—N(R⁵)₂, —C(O)—R⁶—N(R⁵)₂, —N(R⁵)—C(NR⁵)—N(R⁵)₂, —N(R⁵)—C(O)—N(R⁵)₂ and —N(R⁵)—C(O)—R⁶—N(R⁵)₂ where each R⁵ and R⁶ are as defined above.

“Aralkyl” refers to a radical of the formula —RₐR_{b} where Rₐ is an alkyl radical as defined above and R_{b} is one or more aryl radicals as defined above, e.g., benzyl, diphenylmethyl and the like. The aryl radical(s) may be optionally substituted as described above.

“Aralkoxy” refers to a radical of the formula —OR_{d} where R_{d} is an aralkyl radical as defined above, e.g., benzyloxy, and the like. The aryl radical may be optionally substituted as described above.

“Aralkenyl” refers to a radical of the formula —R_{c}R_{b} where R_{c} is an alkenyl radical as defined above and R is one or more aryl radicals as defined above, e.g., 3-phenylprop-1-enyl, and the like. The aryl radical(s) and the alkenyl radical may be optionally substituted as described above.

“Alkylene chain” refers to a straight or branched divalent hydrocarbon chain consisting solely of carbon and hydrogen, containing no unsaturation and having from one to eight carbon atoms, e.g., methylene, ethylene, propylene, n-butylene, and the like. The alkylene chain may be optionally substituted by one or more substituents selected from the group consisting of aryl, halo, hydroxy, alkoxy, haloalkoxy, cyano, nitro, mercapto, alkylthio, cycloalkyl, —N(R⁵)₂, —C(O)OR⁵, —C(O)N(R⁵)₂ or —N(R⁵)—C(O)—R⁵ where each R⁵ is as described above. The alkylene chain may be attached to the rest of the molecule through any two carbons within the chain.

“Alkenylene chain” refers to a straight or branched divalent hydrocarbon chain consisting solely of carbon and hydrogen, containing at least one double bond and having from two to eight carbon atoms, e.g., ethenylene, prop-1-enylene, but-1-enylene, pent-1-enylene, hexa-1,4-dienylene, and the like. The alkenylene chain may be optionally substituted by one or more substituents selected from the group consisting of aryl, halo, hydroxy, alkoxy, haloalkoxy, cyano, nitro, mercapto, alkylthio, cycloalkyl, —N(R⁵)₂, —C(O)OR⁵, —C(O)N(R⁵)₂ or —N(R⁵)—C(O)—R⁵ where each R⁵ is as described above. The alkenylene chain may be attached to the rest of the molecule through any two carbons within the chain.

“Cycloalkyl” refers to a stable monovalent monocyclic or bicyclic hydrocarbon radical consisting solely of carbon and hydrogen atoms, having from three to ten carbon atoms, and which is saturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents independently selected from the group consisting of alkyl, aryl, aralkyl, halo, haloalkyl, hydroxy, alkoxy, haloalkoxy, cyano, nitro, mercapto, alkylthio, cycloalkyl, —N(R⁵)₂, —C(O)OR⁵, —C(O)N(R⁵)₂ or —N(R⁵)—C(O)—R⁵ where each R⁵ is as defined above.

“Cycloalkylene” refers to a stable divalent monocyclic or bicyclic hydrocarbon consisting solely of carbon and hydrogen atoms, having from three to ten carbon atoms, and which is saturated and attached to the rest of the molecule by two single bonds, e.g., cyclopropylene, cyclobutylene, cyclopentylene, cyclohexylene, decalinylene and the like. Unless otherwise stated specifically in the specification, the term “cycloalkylene” is meant to include cycloalkylene moieties which are optionally substituted by one or more substituents independently selected from the group consisting of alkyl, aryl, aralkyl, halo, haloalkyl, hydroxy, alkoxy, haloalkoxy, cyano, nitro, mercapto, alkylthio, cycloalkyl, —N(R⁵)₂, —C(O)OR⁵, —C(O)N(R⁵)₂ or —N(R⁵)—C(O)—R⁵ where each R⁵ is as defined above.

“N-heterocyclyl” refers to a stable 3- to 15-membered ring radical which consists of carbon atoms and from one to five heteroatoms selected from the group consisting of nitrogen, oxygen and sulfur wherein at least one of the heteroatoms is a nitrogen. For the purposes of this invention, the N-heterocyclyl radical may be a monocyclic, bicyclic or a tricyclic ring system, which may include fused or bridged ring systems; and the nitrogen, carbon or sulfur atoms in the N-heterocyclyl radical may be optionally oxidized; the nitrogen atom may be optionally quaternized; and the N-heterocyclyl radical may be partially or fully saturated or aromatic. The N-heterocyclyl radical may be attached to the main structure at any heteroatom or carbon atom which results in the creation of a stable compound. Examples of such N-heterocyclyl radicals include, but are not limited to, azepinyl, azetidinyl, benzimidazolyl, benzoxazolyl, carbazolyl, decahydroisoquinolyl, quinuclidinyl, imidazolyl, imidazolinyl, imidazolidinyl, isothiazolidinyl, indolyl, isoindolyl, indolinyl, isoindolinyl, indolizinyl, isoxazolyl, isoxazolidinyl, morpholinyl, benzothiadiazolyl, oxadiazolyl, octahydroindolyl, octahydroisoindolyl, 2-oxopiperazinyl, 2-oxopiperidinyl, 2-oxopyrrolidinyl, 2-oxoazepinyl, oxazolyl, oxazolidinyl, perhydroazepinyl, piperidinyl, piperazinyl, 4-piperidonyl, phenazinyl, phenothiazinyl, phenoxazinyl, phthalazinyl, pteridinyl, purinyl, pyrrolyl, pyrrolidinyl, pyrazolyl, pyrazolidinyl, pyridinyl, pyrazinyl, pyrimidinyl, pyridazinyl, quinazolinyl, quinoxalinyl, quinolinyl, quinuclidinyl, isoquinolinyl, thiazolyl, thiazolidinyl, thiadiazolyl, triazolyl, tetrazolyl, tetrahydroisoquinolyl, thiomorpholinyl, thiomorpholinyl sulfoxide, and thiomorpholinyl sulfone. The carbon atoms in the N-heterocyclyl radical may be optionally substituted by alkyl, halo, nitro, cyano, haloalkyl, haloalkoxy, mercapto, alkylthio, phenyl, cycloalkyl, —OR⁵, —N(R⁵)₂, —R⁶—N(R⁵)₂, —N(R⁵)—C(O)OR⁵, —R⁶—N(R⁵)—C(O)OR⁵, —N(R⁵)—C(O)—R⁵, —R⁶—N(R⁵)—C(O)—R⁵, —C(O)OR⁵, —R⁶—C(O)OR⁵, —C(O)—N(R⁵)₂, —R⁶—C(O)—N(R⁵)₂, —C(O)—R⁶—N(R⁵)₂, —N(R⁵)—C(NR⁵)—N(R⁵)₂, —N(R⁵)—C(O)—N(R⁵)₂ and —N(R⁵)—C(O)—R⁶—N(R⁵)₂ where each R⁵ and R⁶ are as defined above in the

The nitrogen atoms in the N-heterocyclyl may be optionally substituted by —C(NR⁵)—N(R⁵)₂, —C(NR⁵)—R⁶, —C(O)—N(R⁵)₂ or —C(O)—R⁵—N(R⁵)₂ where each R⁵ and R⁶ are as defined above. Preferred N-heterocyclyl radicals are piperidinyl, tetrahydrosoquinolinyl, or benzothiadiazolyl.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. “Haloalkoxy” refers to a radical of the formula —OR_{c} where R_{c} is an haloalkyl radical as defined above, e.g., trifluoromethoxy, difluoromethoxy, trichloromethoxy, 2,2,2-trifluoroethoxy, 1-fluoromethyl-2-fluoroethoxy, 3-bromo-2-fluoropropoxy, 1-bromomethyl-2-bromoethoxy, and the like.

The LRH-1 agonist of the invention can preferably be a physiologically functional derivative of the 9-substituted bicycle [3.3.0] octane derivative such as a substituted cis-bicyclo[3.3.0]-oct-2-ene derivative described herein.

As used herein, the term “physiologically functional derivative” refers to any pharmaceutical acceptable derivative of a LRH-1 agonist, for example, an ester or an amide. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

It will also be appreciated by those skilled in the art that the LRH1 agonist may also be utilized in the form of a pharmaceutically acceptable salt or solvate thereof. The physiologically acceptable salts include conventional salts formed from pharmaceutical acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts. References hereinafter to a LRH1 agonist include both compounds and their pharmaceutical acceptable salts and solvates.

A preferred example of a LRH-1 agonists is a 9-substituted bicycle [3.3.0] octane derivative such as a substituted cis-bicyclo[3.3.0]-oct-2-ene derivative of Formula II with an EC50 of 430 nM (Whitby et al. (2006), J. Med. Chem. 49:6652-6655) that is applied in the uses and methods of the invention.

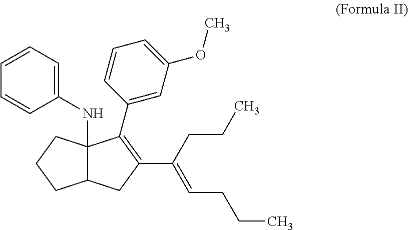 [SMILES: CCC/C=C(\CCC)C1=C(c2cccc(OC)c2)C2(Nc3ccccc3)CCCC2C1]

Another preferred example of a LRH-1 agonists is a 9-substituted bicycle [3.3.0] octane derivative such as a substituted cis-bicyclo[3.3.0]-oct-2-ene derivative of Formula III with an pEC50 of 6.6 (Whitby et al. (2011), J. Med. Chem. 54: 2266-2281) that is applied in the uses and methods of the invention.

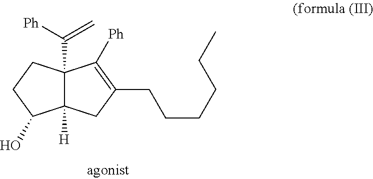 [SMILES: C=C(c1ccccc1)[C@@]12CC[C@@H](O)[C@@H]1CC(CCCCCC)=C2c1ccccc1]

Preferably, LRH-1 agonists applied in the present invention have the same physico-chemical and/or pharmacological properties as the compound of Formula II. Accordingly, LRH-1 agonists applied in the present invention have preferably an EC50 equal or below about 430 nM in a fluorescence resonance energy transfer (FRET)-based assay to detect the interaction of the ligand binding domain of LRH-1. This screen is described in detail in the Supporting Information under Biological Methods “FRET Assays for LRH-1 and SF-1” of Whitby et al. Specifically, the screen utilizes a ligand mediated co-factor interaction between the purified bacterial expressed ligand binding domain of human LRH1 (amino acids 300-541 of the sequence deposited with UniProt Accession No. O 00482, EMBL Accession No. AAC78727.1 or GenBank Accession No. AAD37378.1) and a peptide derived from TIF2 (amino acids 737-757, i.e. B-QEPVSPKKKENALLRYLLDKDDTKD-CONH2). Detection of the associated complex was measured by time resolved fluorescence energy transfer (TR-FRET). 20 nM of the purified ligand binding domain of LRH1 labeled with biotin was mixed with stoichiometric amounts of APC labeled streptavidin (Molecular Probes) in assay buffer (50 mM MOPS pH 7.5, 50 mM NaF, 50 μM CHAPS, 1 mM EDTA, 10 mM DTT). 40 nM of biotinylated TIF2 peptide was mixed with a 0.5 equivalents of europium labeled streptavidin (Wallac Inc) in assay buffer. Each mixture was blocked with a 5 fold molar excess of biotin and allowed to equilibrate for 15 min. The receptor-APC complex and peptide-europium complex were mixed together at a ratio of 1 to 2, allowed to equilibrate for at least 30 min then added to concentrations of test compound diluted in 100% DMSO. After a 2 hr equilibration, the time-resolved fluorescent signal was quantitated using a ViewLux Ultra HTS Microplate Imager (PerkinElmer Life and Analytical Sciences, Inc. Wellesley, Mass., USA) in time resolved mode. The EC50s of the test compound was estimated from a plot using the ratio of fluorescence values collected at 671 nm to fluorescence values collected 618 nM versus concentration of test compound added. Test compounds that increased the affinity of the receptors for the peptide yielded an increase in fluorescent signal. The above being said, BL-001 can serve as the “reference” LRH-1 agonist when further LRH-1 agonists having an EC50 equal or below about 430 nM in a fluorescence resonance energy transfer (FRET)-based assay value are isolated in accordance with the teaching disclosed herein.

The EC50 is defined as the concentration of agonist that provokes a response half way between the baseline response and maximum response on a dose response curve where the X-axis plots concentration of an agonist and the Y-axis plots ion current. An inverse agonist (also called negative antagonist) is a drug which acts at the same receptor as that of an agonist, yet produces an opposite effect. A partial agonist is an endogenous substance or a drug that also provokes physiological or a pharmacological response but, the maximum response is less than the maximum response to a full agonist, regardless of the amount of drug applied. In the case of LRH-1, partial agonists have preferably EC50s higher than 430 nM in the above-described FRET assay.

The reduction of pancreatic β-cell mass (BCM) plays a critical role in the pathogenesis of both Type I and Type II diabetes. Autopsy data indicate that patients with Type II diabetes often manifest over 50% reduction in BCM compared to non-diabetic individuals. It is assumed that the continued progressive loss of BCM is responsible for the deterioration of glycemic control and for the ultimate failure of several classes of oral hypoglycemia agents. Thus, the present invention aims at providing means and methods for preventing progressive loss of pancreatic β-cell mass. Indeed, the present inventors elucidated a way to preserve and/or restore pancreatic β-cells which may otherwise be subject to cell death both in type I and type II DM. Accordingly, the elucidation of a way to prevent the progressive loss of pancreatic β-cells allows the application of preventive medicine regarding type I and/or type II DM—an aspect that was not disclosed up to the present invention.

Already a slight reduction in the progressive loss of BCM is beneficial for a subject who suffers from the loss of pancreatic β-cells, since the pancreatic β-cells can exert their function for a longer period of time, thereby a subject is, for example, not that fast (fully) dependent on externally administered insulin.

However, in order to measure a progressive loss, it is necessary to have available means and methods for determining/measuring β-cell mass in a subject.

Souza et al. in J. Clin. Invest. 116: 1506-1513 (2006) disclose a positron emission tomography (PET). In detail, a positron emission tomography (PET)-based quantitation of pancreatic radiolabeled vesicular monoamine transporter Type 2 (VMAT2) receptors in diabetic rats is a non-invasive way to measure BCM. WO2007/005283 discloses non-invasive methods for determining BCM in the pancreas of a subject by administering to the subject an effective amount of a VMAT2-specific radioligand; obtaining at least one computerized image of at least a portion of the pancreas of the subject; and quantitatively analyzing the computerized image in order to determine the beta cell mass in the pancreas of the subject.

Measuring insulin or C-peptide or other blood biochemistry tests as well as the need for (external) insulin, levels of fasting glucose and/or the mean amplitude of glycemic excursions over time are known measures in the art to determine BCM.

More specifically, apart from insulin, beta cells release C-peptide. Accordingly, measuring the levels of C-peptide can give a practitioner an idea of the viable beta cell mass. (Hoogwerf and Goetz (1983), J Clin Endocrinol Metab 56: 60-67).

In the alternative, the number of pancreatic β-cells may be determined by measuring the circulation levels of the CFC 1 protein in plasma, serum, or whole blood obtained from a mammalian subject in accordance with the teaching provided by WO 2009/131852. In particular, it has recently been observed that CFC 1 is highly and selectively expressed in primate pancreatic islet cells and that protein expression of CFC 1 co-localizes with insulin-producing beta cells in the pancreas. Additionally, it has been discovered that CFC 1 protein can be shed from cultured human islets in a glucose-independent manner. Therefore, it was concluded that CFC 1 protein is a novel biomarker for the measurement of pancreatic islet beta-cell mass.

Therefore, the level of CFC 1 can be used for monitoring pancreatic islet beta-cell mass in a method comprising detecting and measuring the amount of CFC 1 protein in the plasma, serum, or whole blood of a subject, particularly a subject that suffers from or may be prone to progressive loss of pancreatic β-cells pancreatic cells. The level of CFC 1 protein may be measured by any known immunological technique/assay applying antibodies that are specific for the CFC 1 protein.

Thus, an immunoassay method to measure pancreatic β-cell mass in a subject is envisaged using an antibody, which binds to CFC 1 protein, the method comprising the steps of (a) obtaining a biological sample from the subject; (b) contacting the biological sample with an antibody specific for CFC 1 protein under conditions which allow binding of the CFC 1 protein to the antibody; and (b) detecting the presence of the CFC 1 protein in the biological sample, wherein the amount of the CFC 1 protein in the sample provides a measurement of the pancreatic β-cell mass in the subject.

In particular, the higher the amount of CFC 1 protein the higher is the pancreatic β-cell mass and the lower the amount of CFC 1 protein the lower is the pancreatic β-cell mass. In general, the biological sample is whole blood, serum, or plasma. In a further aspect, the method further comprises the step of (c) comparing the amount of the CFC 1 in the biological sample to a control value for CFC 1 protein in the subject. The term “control value” as used herein refers to a basal level of CFC 1 that is present in the subject obtained prior to the commencement of a treatment regime or transplantation therapy.

“Prevention” or “preventing” the progressive loss of pancreatic β-cells refers to prophylactic or preventative measures, wherein the object is to prevent or slow down (lessen) a pathologic condition, i.e., loss of pancreatic β-cells. Slowing down the progressive loss of pancreatic β-cells includes the reduction of the number of pancreatic β-cells that are lost. Preferably, reduction includes at least a reduction of the number of pancreatic β-cells that would otherwise (i.e. without the administration of the LRH-1 agonist of the invention) be lost of at least 5, 10, 20, 30, 40, 50, 60, 70, 80, 90 or 100%. The number of pancreatic β-cells is indicative of the pancreatic β-cell mass (BCM). Accordingly, the term “number of pancreatic β-cells” and “pancreatic β-cell mass (BCM)” can be interchangeably used. The reduction of BCM measured/determined by means and methods known in the art, preferably those described herein.

Those in need of a prophylaxis/prevention include those already with the pathologic condition and/or those prone to have the pathologic condition and/or those in whom the disorder is to be prevented. Accordingly, those already with the pathologic condition are treated and thus subject to a treatment with an LRH-1 agonist to prevent further loss of pancreatic β-cells. Hence, the term “prevention” also includes the treatment of the pathological condition (progressive loss of pancreatic β-cells).

A subject is successfully “prevented” if, after receiving a prophylactic amount of an LRH-1 agonist according to the teaching of the invention, the subject shows observable and/or measurable slowing down or reduction in the progressive loss of pancreatic β-cells.

The term “prophylactic effective amount” means any amount which, as compared to a corresponding subject who has not received such amount, results in prevention or amelioration of the loss of pancreatic β-cells. The term also includes within its scope an amount effective to treat progressive loss of pancreatic β-cells, i.e., a “therapeutically effective amount”.

The exact dose will depend on the purpose of the prevention/treatment, and will be ascertainable by one skilled in the art using known techniques. As is known in the art, adjustments for age, body weight, general health, sex, diet, drug interaction and the severity of the pathological condition may be necessary, and will be ascertainable with routine experimentation by those skilled in the art. The prophylactic effect of the LRH-1 agonist of the invention is additionally detectable by all established methods and approaches which will indicate a prophylactic (and thus therapeutic) effect. It is, for example, envisaged that the prophylactic effect is detected by determining BCM by means and methods known in the art, preferably those described herein.

“Progressive” loss means that the number of pancreatic β-cells (pancreatic β-cell mass (BCM)) gets more and more, i.e. continuously reduced. Continuously, however, does not mean that the same number of pancreatic β-cells is lost over a period of time. Rather, “continuously” means that the reduction cannot be constantly stopped, since it proceeds due to a pathological condition.

The term “pancreatic β-cell” includes any cell of a mammalian organ or tissue, capable of exerting β-cell function, in particular a β-cell produces insulin in vivo, including synthetic or semi-synthetic cells, or transgenic cells. It also includes cells from foetal pancreas or stem cells that have done a first differentiation in endodermic cells. Preferably, pancreatic β-cells are cells in the pancreas in areas called the islets of Langerhans. They make up 65-80% of the cells in the islets. Accordingly, the pancreatic β-cells are preferably pancreatic islet β-cells.

It is assumed that a β-cell mass (BCM) of about 50% of the normal BCM, i.e., the BCM of a subject not suffering from progressive loss of β-cells leads to pathological conditions (Matveyenko and Butler (2008), Diabetes, Obesity and Metabolism 10:23-31).

Specifically, a progressive loss of β-cells leads, inter alia, to impaired plasma insulin levels (when evaluated directly in the portal vein). Consequently it also leads to a decreased level of CFC 1 and/or further symptoms as described herein elsewhere. Typically, a progressive loss of β-cells through cell death, preferably through apoptosis, leads to impaired functional β-cell mass. Next, decompensation of glucose control follows when the pattern of portal vein insulin secretion is sufficiently impaired to cause hepatic insulin resistance. Finally, adverse consequences of glucose toxicity accelerate β-cell dysfunction and insulin resistance.

Accordingly, it is envisaged that by applying the LRH-1 agonist of the invention to β-cells the functional β-cell mass can be maintained at a level above 50% of the normal BCM so that β-cell function is essentially not impaired. “Essentially not” means that β-cell function is impaired to an extent of not more than about 50, 40, 30, 20, 10, 5%, with 20, 10 or 5% being preferred.

β-cell function is, inter alia, characterized by the production and regulated release of insulin, a hormone that controls the level of glucose in the blood. There is a baseline level of glucose maintained by the liver, but it can respond quickly to spikes in blood glucose by releasing stored insulin while simultaneously producing more. The response time is fairly quick, taking less than 10 minutes.

Apart from insulin, β-cells release C-peptide, a byproduct of insulin biosynthesis, into the bloodstream in equimolar quantities. C-peptide helps to prevent neuropathy and other symptoms of diabetes related to vascular deterioration. Measuring the levels of C-peptide can give a practitioner an idea of the viable beta cell mass as described herein elsewhere. β-cells also produce amylin, also known as IAPP, islet amyloid polypeptide. Amylin functions as part of the endocrine pancreas and contributes to glycemic control. Amylin's metabolic function is now somewhat well characterized as an inhibitor of the appearance of nutrient (especially glucose) in the plasma. It thus functions as a synergistic partner to insulin. Whereas insulin regulates long term food intake, increased amylin decreases food intake in the short term.

The progressive loss of pancreatic β-cells, preferably pancreatic islet β-cells, is preferably due to cell death of pancreatic islet β-cells.

“Cell death” includes death of pancreatic β-cells in any form, for example, mediated by programmed cell-death (PCD) or necrosis.

Preferably, the cell death of β-cells is due to apoptosis. Apoptosis is one of the main types of (PCD). It is a process of suicide by a cell in a multi-cellular organism and involves an orchestrated series of biochemical events leading to a characteristic changes in cell morphology and finally death. These changes include blebbing, loss of cell membrane asymmetry and attachment, cell shrinkage, nuclear fragmentation, chromatin condensation, and chromosomal DNA fragmentation. Apoptosis differs from necrosis, in which the cellular debris can damage the organism.

In the context of the present invention the term “necrosis” includes morphological changes of cells, preferably pancreatic β-cells leading finally to cell death. Necrosis is, inter alia, characterized for example by “leakiness” of the cell membrane, i.e., an increased permeability which also leads to an efflux of the cell's contents and an influx of substances perturbing homeostasis and ion equilibrium of the cell, DNA fragmentation and, finally, to the generation of granular structures originating from collapsed cells, i.e. cellular debris. Typically, necrosis results in the secretion of proteins into the surrounding which, when occurring in vivo, leads to a pro-inflammatory response. Methods for the determination whether a cell is necrotic or not are known in the prior art. It is not important which method the person skilled in the art chooses since various methods are known.

However, it is important to distinguish between an apoptotic cell undergoing the so called programmed cell death and a necrotic cell. Necrotic pancreatic β-cells cells in accordance with the present invention can be determined, e.g., by light-, fluorescence or electron microscopy techniques, using, e.g., the classical staining with trypan blue, whereby the necrotic cells take up the dye and, thus, are stained blue, or distinguish necrotic cells via morphological changes including loss of membrane integrity, disintegration of organelles and/or flocculation of chromatin. Other methods include flow cytometry, e.g., by staining necrotic cells with propidium iodide. Apoptotic cells can be determined, e.g., via flow-cytometric methods, e.g., attaining with Annexin V-FITC, with the fluourchrome:Flura-red, Quin-2, with 7-amino-actinomycin D (7-AAD), decrease of the accumulation of Rhodamine 123, detection of DNA fragmentation by endonucleases; TUNEL-method (terminal deoxynucleotidyl transferase caused X-UTP nick labelling), via light microscopy by staining with Hoechst 33258 dye, via Western blot analysis, e.g., by detecting caspase 3 activity by labelling the 89 kDa product with a specific antibody or by detecting the efflux of cytochrome C by labelling with a specific antibody, or via agarose gel DNA-analysis detecting the characteristic DNA-fragmentation by a specific DNA-ladder or by ELISA detecting nucleosome released from DNA fragmentation.

In the context of the cell death of pancreatic β-cells, apoptosis of pancreatic β-cells is preferably stress-induced. Stress includes any kind of stress such as heat, cold, toxic concentrations of metabolites or other extracellular stimuli which could cause cell death. Preferably, apoptosis of pancreatic β-cells is due to pro-inflammatory cytokines. Generally, a proinflammatory cytokine is a cytokine which promotes systemic inflammation. Typical proinflammatory cytokines are TNF-α, IL-1β and IFN-γ which are thus the ones that are deemed responsible to cause stress-induced apoptosis of pancreatic β-cells. Accordingly, it is preferably envisaged that TNF-α, IL-1β or IFN-γ alone or a combination of TNF-α and IL-1β, TNF-α and IFN-γ or IL-1β and IFN-γ or a combination of TNF-α, IL-1β or IFN-γ causes stress-induced apoptosis of pancreatic β-cells.

In a preferred embodiment, the use of a LRH-1 agonist in the prevention of progressive loss of pancreatic β-cells preferably facilitates the preservation or restoration of pancreatic β-cells, preferably pancreatic islet β-cells. Accordingly, it is an aspect of the invention to apply an LRH-1 agonist for use in the preservation or restoration of pancreatic β-cells, preferably pancreatic islet β-cells.

By “preservation” of pancreatic β-cells is meant that the pancreatic β-cells are essentially not impaired in β-cell function. Accordingly, it is this envisaged that by applying a LRH-1 agonist pancreatic β-cells are essentially maintained in the state as they are, i.e., they are essentially preserved. Preferably, preservation also includes that pancreatic β-cells are protected against cell death, in particular cell death due to apoptosis, preferably stress-induced apoptosis, wherein the stress is preferably induced by pro-inflammatory cytokines as described herein.

Similarly, the preservation of pancreatic β-cells is applicable for the preservation of pancreatic β-cells in vitro. For example, when pancreatic β-cells are stored in vitro before they are grafted into a recipient. “Storing” includes culturing and/or washing of pancreatic β-cells.

“Pancreatic β-cell restoration” refers generally to restoration of normal function of a pancreatic β-cell, in particular the restoration of insulin production

In a further preferred embodiment, the use of a LRH-1 agonist in the prevention of progressive loss of pancreatic β-cells preferably facilitates the prevention or treatment of type I diabetes. Accordingly, it is an aspect of the invention to apply an LRH-1 agonist for use in the prevention or treatment of type I diabetes.

Type 1 diabetes results from the impairment of pancreatic β-cells to produce insulin, and thus requires a subject to be administered insulin. Up to now, it is assumed that type I DM is caused by an autoimmune reaction against pancreatic β-cells. Yet, the causative auto-antigen is still subject to speculation. The present inventors, in contrast to the prior art elucidated that, in particular, mainly pro-inflammatory cytokines contribute to cell death of pancreatic β-cells leading to a progressive loss of this cell type that, however, can be prevented by agonizing LRH-1. This effect was not recognized in the prior art such as in WO 2009/067182. In that application it is suggested treating diabetes by a phosphatidlycholine. However, the inventors of that application merely observed in a murine diabetes model (resembling type II DM) that a phosphatidycholin which acts on LRH-1 facilitates secretion of insulin. Thus, these inventors showed that agonizing LRH-1 increases insulin secretion. Yet, these inventors did not recognize the “true” effect that phosphatidylcholin is having, i.e., the prevention of progressive loss of pancreatic β-cells.

Diabetes is a frequent chronic disease that reduces quality of life and increases the risk for life threatening complications despite current treatment. Its economic burden is estimated at 15% of health care expenses. The disease can appear at all ages. When diagnosed under age 40, it mostly presents as the Type 1 form that is caused by massive loss of insulin-producing beta cells following an inflammatory and immune process. An insufficient beta cell mass is also a pathogenic factor in many Type 2 diabetic patients. Restoring and preserving the beta cell mass is thus a major goal as it may cure diabetes, in particular, type I DM.

Diabetes mellitus can be of Type 1 (also referred to herein sometimes as Type I) or Type 2 (also referred to herein sometimes as Type II). Type 1 diabetes is believed to be an autoimmune disease resulting in destruction of the pancreatic β cells which means the subject is unable to manufacture any insulin. Type 1 diabetes mellitus is characterized by loss of the insulin-producing beta cells of the islets of Langerhans in the pancreas leading to insulin deficiency. This type of diabetes can be further classified as immune-mediated or idiopathic. The majority of Type 1 diabetes is of the immune-mediated nature, where beta cell loss is a T-cell mediated autoimmune attack. There is no known preventive measure against Type 1 diabetes, which causes approximately 10% of diabetes mellitus cases in North America and Europe. Most affected people are otherwise healthy and of a healthy weight when onset occurs. Sensitivity and responsiveness to insulin are usually normal, especially in the early stages. Type 1 diabetes can affect children or adults but was traditionally termed “juvenile diabetes” because it represents a majority of the diabetes cases in children.

Type 2 diabetes is a more complicated condition and can result from a number of associated ailments but commonly involves β-cell dysfunction and resistance to the metabolic actions of insulin. For example, Type 2 diabetes is associated with age, obesity, a sedentary life style. An associated condition is called Metabolic Syndrome which may predispose subjects to Type 2 diabetes. The symptoms associated with this syndrome are high blood pressure, dyslipidemia, increased body fat deposition and cardiovascular disease. Type 2 diabetes mellitus is characterized by reduced insulin secretion and increased insulin resistance. The defective responsiveness of body tissues to insulin is believed to involve the insulin receptor. However, the specific defects are not known. DM due to a known defect are classified separately. Type 2 diabetes is the most common type. In the early stage of Type 2 diabetes, hyperglycemia can be reversed by a variety of measures and medications that improve insulin secretion, insulin sensitivity or reduce glucose production by the liver. As the disease progresses, therapeutic replacement of insulin may sometimes become necessary in certain patients

In a yet further preferred embodiment, the use of a LRH-1 agonist in the prevention of progressive loss of pancreatic β-cells preferably facilitates the increment of the survival of pancreatic β-cells, preferably pancreatic islet β-cells.

Accordingly, it is an aspect of the invention to apply an LRH-1 agonist for use in the increment of survival of pancreatic β-cells, preferably pancreatic islet β-cells.

By “increment of the survival” of pancreatic β-cells is meant an increase of some amount, either fixed or variable, of the survival of pancreatic β-cells so that pancreatic β-cells can withstand cell death, in particular cell death by stress-induced apoptosis due to proinflammatory cytokines. The increment includes that pancreatic β-cells are more resistant and thus protected against cell death than pancreatic β-cells which are not treated with a LRH-1 agonist in accordance with the teaching of the invention. Preferably, pancreatic β-cells are at least 5, 10, 20, 30, 40, 50, 60, 70, 80, 90 or even 100% resistant against cell death if they are treated with a LRH-1 agonist in comparison to pancreatic β-cells not treated with a LRH-1 agonist.

The same is applicable for the increment of the survival of a pancreatic β-cell graft, preferably a pancreatic islet β-cell graft. Thus, in another preferred embodiment, the use of a LRH-1 agonist in the prevention of progressive loss of pancreatic β-cells preferably facilitates the increment of the survival of a pancreatic β-cell graft, preferably a pancreatic islet β-cell graft. Accordingly, it is an aspect of the invention to apply an LRH-1 agonist for use in the increment of survival of a β-cell graft, preferably a pancreatic β-cell graft, more preferably a pancreatic islet β-cells.

A “β-cell graft” can be any type of insulin-producing tissue transplant, preferably the β-cells are pancreatic β-cells, more preferably pancreatic islet β-cells, preferably from a subject as described herein. Grafting includes procedure that is suitable to transfer insulin-producing tissue from a donor to a recipient.

More specifically, “grafting” refers to inoculating, implanting, transplanting or transferring a β-cell graft in recipient subject.

It is envisaged that grafting includes procedures wherein the donor (graft) tissue is affected by, or at risk of, failure or rejection by the recipient host's immune system. In particular, it is envisaged that the invention can be used in any context wherein the donor tissue is not histocompatible (MHC-compatible) with the recipient host. Thus, in addition to autologous or syngeneic donor tissue, it is envisaged that the invention can be used with allogeneic or xenogeneic donor tissue. The donor tissue can be derived, by conventional means, from a volunteer or other living donor, or from a cadaveric donor. In one embodiment, the donor is as histocompatible as practicable with the recipient host. For example, where the recipient host is a human, autologous and allogeneic donor tissue is used. In another embodiment, the donor tissue can be obtained from a heterologous species (in which case it is referred to as a heterograft), such as a non-human primate, e.g., a chimpanzee or a baboon, or a member of the porcine species, e.g., a pig.

In some embodiments, the donor islet cells comprise a part, portion or biopsy of a donor pancreas which comprises insulin-producing cells.

If a cadaveric donor is used, the pancreas is preferably exposed to cold ischemic conditions for no more than about eight hours. In still other embodiments, the donor islet cells comprise isolated or suspended islets or islet cells, including cells withdrawn or excised from a fetal or adult donor, cells maintained in primary culture, or an immortalized cell line. Appropriate means for preparing donor islets or islet cell suspensions from whole pancreata are well known (see, e.g., Ricordi et al., (1988), Diabetes, 37: 413; Tzalcis et al. (1990), Lancet, 336: 402; Linetsky et al. (1997), Diabetes, 46: 1120. Appropriate pancreata are obtained from donors essentially free of defects in blood glucose homeostasis.

Other sources of insulin-producing cells include islet progenitor cells, such as fetal cells, optionally expanded in primary culture. Any appropriate cell type can be used, however, including cells harbouring exogenous genetic material encoding an expressible insulin gene. Thus, the invention encompasses the use of transfected or transformed host cells, which have been (or are derived from ancestor cells which have been) engineered to express insulin, either constitutively or inducibly (e.g., under control of a glucose-responsive promoter or enhancer). In other embodiments, the invention encompasses the use of pancreatic or other donor cell types derived from a transgenic mammal that has been engineered to include genetic material necessary for the production of insulin in some or all of its body tissues.

The insulin producing tissue (donor tissue) is introduced systemically or locally into the recipient host. For example, isolated, suspended or dispersed insulin-producing cells can be infused intravascularly, or implanted into a desired site, such as a bone marrow cavity, the liver, within the kidney capsule, intramuscularly, or intraperitoneally. In some embodiments, the cells are mitotically competent and produce new tissue of donor origin. In other embodiments, the cells are not mitotically competent, but remain viable in the donor, and produce or express insulin. In any event, an effective amount of insulin-producing cells or tissue is implanted, by which is meant an amount sufficient to attenuate (detectably mitigate) the recipient's defect in glucose metabolism (e.g., hypoglycemia or hyperglycemia). Optimally, the amount is sufficient to restore the recipient's ability to maintain glucose homeostasis, so as to free the recipient from dependence on conventional (e.g., injected or inhaled) insulin replacement therapy.

In some embodiments, the insulin-producing tissue is physically separated (isolated) from surrounding tissues of the recipient by an immunoisolation device. Appropriate devices protect the insulin-producing tissue from most effectors of cellular and humoral immunity, including but not limited to, leukocytes, immunoglobulin and complement. Thus, the immunoisolation device generally provides a semipermeable barrier, such as a membrane, having a pore size sufficient to prevent diffusion therethrough of molecules more massive than about 50 to 100 kD. The barrier defines an isolation chamber in which the insulin-producing tissue is disposed, and is free of any sites at which the insulin-producing tissue can physically contact cells or tissues external to the barrier. Any conventional device, envelope, capsule or microcapsule can be used, including single- or double-walled alginate microcapsules (e.g., as described in U.S. Pat. No. 5,227,298). Other conventional microcapsules include alginate polylysine microcapsules, chemically crosslinked alginate microcapsules, and capsules formed of other biocompatible polymers, formed into a structurally sound immunoisolation device of any desired shape or size (see, e.g., Jaink et al. (1996), Transplantation 61: 4).

In a further aspect, the invention relates to a LRH-1 agonist for use in a method of transplanting pancreatic β-cells, preferably pancreatic β-cells, more preferably pancreatic islet β-cells comprising:
(a) isolating β-cells, preferably pancreatic β-cells, more preferably pancreatic islet β-cells from a donor subject;
(b) cultivating said cells in the presence of an LRH-1 agonist; and
(c) transplanting said cells into a recipient subject.

The transplantation method can be used for any mammalian recipient subject of a β-cell graft, in particular a pancreatic β-cell graft, more particularly a pancreatic islet β-cell graft, or any mammal in need of such al graft. Recipient subjects (also referred to as recipients or hosts) accordingly are afflicted with, or at risk of, a defect in metabolic control of blood glucose metabolism (glucose homeostasis). For example, the recipient can be hyper- or hypo-glycemic.

Isolation of pancreatic β-cells is described, for example, in (Parnaud, 2008)

The cultivation step in the presence of an LRH-1 agonist is done to increase viability, decrease death and improve functional islet mass prior to transplantation. Methods for cultivating pancreatic β-cells is described, for example, in WO 2006/015853.

The term “transplanting” is equivalently used with the term “grafting” as described herein

In an even further preferred embodiment, the use of a LRH-1 agonist in the prevention of progressive loss of pancreatic β-cells preferably facilitates the increment of the performance of pancreatic β-cells, preferably pancreatic islet β-cells.

Accordingly, it is an aspect of the invention to apply an LRH-1 agonist for use in the increment of the performance of pancreatic β-cells, preferably pancreatic islet β-cells.

An “increment of the performance” of pancreatic β-cells is meant as an increase of some amount, either fixed or variable, of the function of pancreatic β-cells so that the function of pancreatic β-cells is increased after the treatment with a LRH-1 agonist, in particular if their function would be impaired without the treatment. Increment of the performance includes that pancreatic β-cells become more active so that they even if they are at likelihood to be subject to cell death, get resistant and may thus escape from cell death in comparison to pancreatic β-cells which are not treated with a LRH-1 agonist in accordance with the teaching of the invention. Preferably, pancreatic β-cells are at least 5, 10, 20, 30, 40, 50, 60, 70, 80, 90 or even 100% resistant against cell death if they are treated with a LRH-1 agonist in comparison to pancreatic β-cells not treated with a LRH-1 agonist. Likewise, the increment of performance includes that pancreatic β-cells become at least 5, 10, 20, 30, 40, 50, 60, 70, 80, 90 or even 100% more active if they are treated with a LRH-1 agonist in comparison to pancreatic β-cells not treated with a LRH-1 agonist.

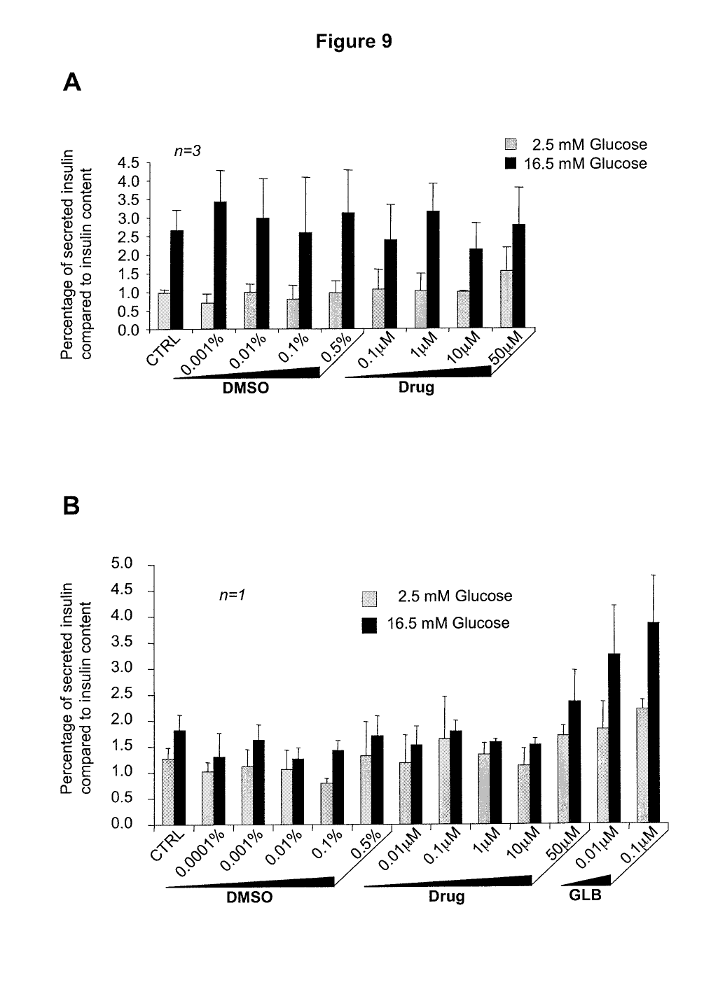

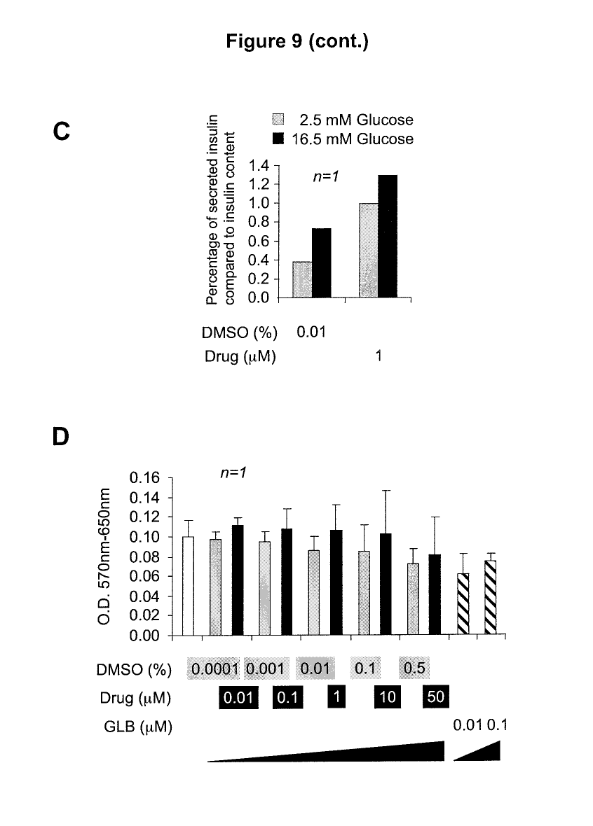

In addition, the present inventors found that LRH-1 agonists stimulate insulin secretion in human normal and diabetic islets. Insulin secretion in response to LRH-1 agonists or glibenclamide in the presence of either 2 mM or 16.5 mM glucose was then studied in islets isolated from a Type 2 diabetic donor. A 40% increase in insulin secretion was evaluated in islets exposed to high glucose alone (FIG. 9A). Accordingly, small variations in both basal and glucose-induced insulin secretion were observed in islets cultured in the presence of DMSO. Interestingly, addition of a LRH-1 agonist resulted in a 40% increase in both basal and stimulated insulin exocytosis while lower concentrations of a LRH-1 agonist had not beneficial impact on secretion in this particular donor. Indeed, a LRH-1 agonist did enhance insulin secretion at low and high glucose in a second donor (FIG. 9B). Glibenclamide prompted an 80% increase in insulin secretion at low glucose. This effect was further accentuated at high glucose reaching 214% as compared to control islets (FIG. 9A). Islet viability was also estimated in the first donor and found slightly reduced at a LRH-1 agonist (FIG. 9C). However, this effect appears to be mediated by DMSO, as increasing the concentration of the vehicle alone resulted in decreased viability. Consistent with a previous report, glibenclamide also impaired cell viability, which could ultimately lead to apoptosis (Maedler, 2005). Thus, similar to glibenclamide, LRH-1 agonists stimulates insulin secretion in diabetic islets both at low and high glucose concentrations.

Accordingly, in another preferred aspect, it is contemplated that a LRH-1 agonist is for use in maintaining insulin secretion.

It is to be understood that the uses of a LRH-1 agonist of the invention are equally applicable in methods of treatment, prevention and/or amelioration, respectively. Similarly, it is to be understood that a LRH-1 agonist of the invention can be used for the preparation of a medicament for the treatment, prevention and/or amelioration of the (pathological) conditions as described herein.

In a yet further aspect, a method of improving the therapy of diabetes is envisaged, said method comprising

(i) contacting β-cells, preferably pancreatic β-cells with a candidate compound;
(ii) contacting said cells of step (i) with one or more proinflammatory cytokines; and
(iii) identifying a compound which is capable of preventing cell death of said cells,
wherein survival of said cells is indicative of an improvement of the therapy of diabetes.

The method allows evaluating a test compound (e.g., potential diabetes drug) against an LRH-1 agonist (such as a LRH-1 agonist of Formula II), by subjecting the test compound and the compound of any of the formulae herein to a subject or medium (e.g., patient, animal model, cell culture, in vitro assay) that provides measure or assessment of the effectiveness of the test compound and the LRH-1 agonist (such as BL-001) in the prevention of progressive loss of pancreatic islet β-cells. The method can further comprise evaluating the results of the compound testing to assess the effectiveness of the test compound as a diabetes drug. The measuring or assessing of the effectiveness of the compounds in these methods can be performed by any number of appropriate techniques and protocols known in the art and readily available.

In the alternative, said method is for screening a candidate compound for treating or ameliorating diabetes.

Candidate compounds are also found among biomolecules including peptides, amino acids, saccharides, fatty acids, steroids, purines, pyrimidines, nucleic acids and derivatives, structural analogs or combinations thereof. Candidate compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The findings of the invention thus pave the way to the objects of the present invention, i.e. the use of a LRH-1 agonist
to prevent the progressive loss of pancreatic β-cells, in particular pancreatic islet β-cells;
to preserve or restore pancreatic β-cells, in particular pancreatic islet β-cells;
to prevent or treat type I diabetes;
to increase the performance of β-cells, in particular pancreatic islet β-cells;
to increase the survival of Langerhans islet cells, including in particular β-cells, more particularly pancreatic islet β-cells;
to increase the performance of β-cells, in particular pancreatic islet β-cells;
to increase the survival of a pancreatic islet graft, including in particular β-cells, more particularly pancreatic islet β-cells;
to increase the in vitro preservation of pancreatic β-cells, in particular pancreatic islet β-cells;
to maintain insulin secretion; and/or
in a method of transplanting pancreatic islet cells with the aim of preserving said cells during their isolation, cultivation and/or transplantation (grafting into the acceptor subject).

The herein described LRH-1 agonists can also be used/applied in methods of the herein described uses by administering a pharmaceutically effectively amount of an LRH-1 agonist described herein to a subject in need thereof. Likewise, the herein described LRH-1 agonists can also be used for the preparation of a medicament/pharmaceutical composition for preventing, ameliorating and/or treating the (medical) conditions as described herein.

The Figures show:

FIG. 1: LRH-1 is expressed in pancreatic islet beta-cells.

(A) Quantitative RT-PCR using RNA purified from isolated rat and human islets, FACS purified beta- and non beta-cells, hepatocytes and intestinal cells were performed to evaluate levels of LRH-1 transcripts. Data are presented as fold change of LRH-1 mRNA levels as compared to islets. (B) Immunofluorescent detection of LRH-1 (red) and insulin (green) in dispersed adult rat islets. (C) Immunofluorescent detection of LRH-1 (green) and insulin (red) in dispersed human islet cells. Nuclei were revealed using DAPI staining. Overlay images show localization of LRH-1 to the nuclei. 40× magnification.

FIG. 2: Doxycycline dose-dependent increases in LRH-1 transcript levels in human islets infected with Ad-hLRH-1.

Human islets were transduced (or not) with Ad-hLRH-1 and Ad-X Tet-On and cultured in the presence of increasing concentrations of doxycycline for 48 h. (A) RNA was subsequently isolated and quantitative RT-PCR was performed to evaluate LRH-1 transcript levels. Data are presented as fold change of LRH-1 mRNA levels as compared to untreated islets. Each value represents the mean±s.e. of three independent experiments performed in duplicate. **P<0.01. (B) Immunofluorescent detection of LRH-1 (green) and insulin (red) as well as DAPI nuclei staining (blue) in dispersed human islet cells 48 h after infection with adenoviruses in the presence or absence of 1 μg/μl doxycycline. Overlay images demonstrate localization of LRH-1 to the nuclei. 40× magnification.

FIG. 3: Overexpression of LRH-1 does not induce proliferation in either INS-1E cells or islets.

Proliferation was measured by BrdU incorporation in INS-1E cells (A), dispersed rat (B) and human (C) islets expressing LRH-1. Cells or islets were transduced (or not) with Ad-hLRH-1 and Ad-X Tet-On, and cultured with or without 1 μg/ml of doxycycline for 48 h. Immunocytochemical detection of BrdU incorporation was performed 24 h after addition of BrdU (10 μM). Data are presented as percentage of BrdU positive cells as compared to the total amount of cells. Each value represents the mean±s.e. of three independent experiments performed in duplicate.

FIG. 4: LRH-1 over expression protects rat islets from either cytokine- or streptozotocin-induced apoptosis.

Islets were co-infected with Ad-hLRH-1 and Ad-X Tet-On and cultured in the presence or absence of 1 ug/ul doxycycline for 48 h. Islets were subsequently treated for 24 h with rat 2 ng/ml IFN-γ, IL-1β and TNF-α to induce apoptosis. Alternatively, islets were treated with 2 mM of streptozotocin. Cell death was measured by the TUNEL assay. More than 700 cells were counted for each condition. ** p<0.01.

FIG. 5: Doxycycline-induced LRH-1 protects human islets against cytokine- and streptozotocin-mediated apoptosis.

Apoptosis was measured by the TUNEL assay in human islets transduced (or not) with Ad-hLRH-1 and Ad-X Tet-On and cultured in the presence or absence of increasing concentrations of doxycycline as presented on the bar graph. Stressed-induced apoptosis was achieved by treating islets for 24 h with 2 ng/mL IFN-γ, IL-1β and TNF-α (A), or 1 and 2 mM of streptozotocin (B). Data are presented as percentage of TUNEL positive cells compared to the total amount of cells. (C) Alternatively, apoptosis was measured using the Cell Death Detection ELISA kit. Data are presented as percentage of apoptotic enrichment compared to untreated islets. Each value represents the mean±s.e. of three independent experiments performed in duplicate. *P<0.05; **P<0.01

FIG. 6: The synthetic LRH-1 agonist of Formula II has no toxic effects on human islets.

Cytotoxicity of increasing concentrations of the LRH-1 agonist of Formula II was measured by MTT assay in human islets and compared to increasing concentrations of vehicle (DMSO) or untreated islets after 24 (grey bars) and 48 h (black bars) of incubation. Data are presented as difference of optic density at 570 and 650 nm. Each value represents the mean±s.e. of one experiment performed in triplicate.

FIG. 7: SHP but not LRH-1 transcript levels are modulated by the LRH-1 agonist of Formula II.

Isolated human islets were incubated with vehicle (DMSO), 17β-estradiol (E2), increasing doses of the LRH-1 agonist of Formula II or a combination of E2 and the LRH-1 agonist of Formula II. RNA was extracted from islets at different time points after treatment and expression levels of LRH-1 (A) and its downstream target, SHP (B) were evaluated by Quantitative RT-PCR. Data are presented as fold change of mRNA levels as compared to time T=Oh. Each value represents the mean±s.e. of 2-3 independent experiments.

FIG. 8: LRH-1 agonist of Formula II protects human islets against cytokine-induced apoptosis.

(A) Protocol describing the apoptosis quantification protocol. Isolated human islets were pre-incubated 24 h with 10 μM of the LRH-1 agonist of Formula II or 0.1% DMSO (vehicle). Additional LRH-1 agonist of Formula II and DMSO treatments were performed at 24 and 48 hours. One hour-post drug treatment (T=1, 25 and 49 h), islets were challenged with either a cocktail of cytokines (2 ng/mL IFN-γ, IL-1β and TNF-α. Apoptosis was measured by the Cell Death Detection ELISA kit at 24 h (B) or 72 h (C). Data are presented as percentage of apoptotic enrichment compared to DMSO-treated islets. Each value represents the mean±s.e. of one experiment performed in duplicate.

FIG. 9: LRH-1 agonist of Formula II stimulates insulin secretion in diabetic islets.

Human non-diabetic (A) and diabetic (B and C) islets were cultured in the presence of increasing concentrations of BL001, DMSO (vehicle) or GLB for 48 h as depicted on the figure. Insulin secretion was assessed in 30 min static incubations in response to 2.5 mM (grey bars) or 16.5 mM glucose (black bars). Insulin released in KRBH was quantified by EIA and expressed as a percentage of total cellular insulin content (A, B and C). (D) Cytotoxicity of increasing concentrations of DMSO (grey bars), BL001 (black bars) and GLB (hatched bars) was measured by MTT assay after 48 h of incubation. Data are presented as difference of optic density at 570 and 650 nm. Results are the mean±s.e. from 3 independent experiments performed in triplicates (A), or from 1 experiment performed in triplicates (D) or in sextuplicates (B and C).

A better understanding of the present invention and of its many advantages will be had from the following examples, offered for illustrative purposes only, and are not intended to limit the scope of the present invention in any way.

Cell Culture

Human breast cancer MCF7 cells were grown in DMEM medium (Invitrogen) supplemented with 10% heat-inactivated foetal calf serum (FCS; Brunschwig AG, Basel, Switzerland), 10 mM HEPES (AppliChem, Darmstadt, Germany), 100 units/ml penicillin and 100 μg/streptomycin (Sigma). Rat insulinoma INS-1E cells (Merglen, 2004) were cultured in RPMI-1640 (Invitrogen, Basel, Calif., USA) supplemented with 10% FCS and other additions as described previously (Asfari, 1992).

Rat and Human Islets

Pancreatic islets from 5-week-old male Wistar rats (Elevage Janvier, Le Genest-St-lsle, France) were isolated by collagenase digestion and hand picked as previously described (Gauthier, 2004). Human islets were kindly obtained from The Cell Isolation and Transplantation Center (Department of Surgery, Geneva; Switzerland), Prof. P. Marchetti (Department of Endocrinology and Metabolism, Metabolic Unit, Cisanello Hospital, Pisa; Italy), Dr. F. Pattou (Cell Therapy for Diabetes-CHRU de Lille, Lille; France) and Dr. E. de Koning (Department of Nephrology, Leiden University Medical Centre, Leiden; Netherlands). Islet preparations were washed, handpicked and subsequently maintained in CMRL-1066 (Gibco) containing 5.6 mM glucose supplemented with 10% FCS, 100 Units/ml penicillin, 100 μg/ml streptomycin and 100 μg/ml gentamycin.

Cell Treatment

INS-1E cells or islets (rat or human) were cultured in phenol red free medium containing 10% Charcoal-stripped FCS for 48 h. The latter removes all traces of endogenous steroids, which may skew results. Subsequently, 0.0001% DMSO (vehicle) and/or 10 nM 17β-estradiol were added to the media. Alternatively human islets were also treated with increasing concentrations of either DMSO (vehicule) or with a combination of estrogen and the LRH-1 agonist of Formula II (SevenHills Pharma, Fort Collins, USA) for various time intervals. For apoptosis studies (FIG. 8A), islets were pre-treated with 0.1% DMSO (vehicule) or with 10 μM of the LRH-1 agonist of Formula II. Twenty-four hours later, DMSO or the LRH-1 agonist of Formula II were added again to the medium and incubated for 1 extra hour prior to the addition of either a cocktail of human cytokines (2 ng/ml IFN-γ, IL-1β and TNF-α). Samples of 50 islets were then collected at 24 and 72 hours. The media was replenished with DMSO and the LRH-1 agonist of Formula II at 24 and 48 hours.

LRH-1 Adenoviral Construction and Transduction

The full-length human LRH-1 cDNA cloned into the expression vector pcDNA-T7tag was kindly provided by Lilly (Hamburg, Germany). Subsequently, LRH-1 cDNA was subcloned into the pTRE-Shuttle2 vector (CLONTECH Laboratories, Inc.). The inducible cassette was transferred into the Adeno-X viral DNA to generate the recombinant adenovirus Ad-hLRH-1. INS-1E cells were seeded at 3×10⁵ cells/ml in 24-well plates whereas 150 islets (rat or human) were used per experimental condition. Cells or islets were subsequently co-infected with Ad-hLRH-1 along with the adenoviral construct harbouring the tetracycline transcriptional activator (Ad-X Tet-On) at a ratio of 2:1 (3.6×10⁷ pfu/ml total viral particles). Cells were washed either 90 minutes (islets) or 3 h (INS-1E) post infection and cultured in fresh media supplemented with the indicated concentration of doxycycline.

Quantitative RT-PCR

Total RNA was extracted using the RNeasy Micro Kit (Qiagen) and 1-2 μg was converted into cDNA with the Superscript II Reverse transcriptase (Invitrogen). Primers were designed using the Primer Express software (Applera Europe, Rotkreuz, Switzerland) and sequences are provided in Table 1. Real time PCR was performed using an ABI 7000 Sequence Detection System (Applera Europe), and PCR products were quantified fluorometrically using the FastStart Universal SYBR Green Master (Roche Diagnostics, Rotkreuz, Switzerland). Three distinct amplifications were performed in duplicate for each transcript, and mean values were normalized to the mean value of the reference mRNA cyclophilin.

### Immunohistochemistry

Control, treated and transduced islets were trypsinized to produce a single cell suspension and then cytospined unto glass slides. Cells were washed with PBS and fixed in 4% paraformaldehyde for 20 min at room temperature. Endogenous and recombinant LRH-1 were visualized by immunohistochemistry using an LRH-1 anti-sera (Abcam, Cambridge, UK). Immunochemical detection of β-cells was performed as previously described (Brun, 2004). Nuclei were stained with DAPI (10 μg/ml; Sigma). Cover slips were mounted using DAKO fluorescent mounting medium and visualized using a Zeiss Axiophot I.

Proliferation and Apoptosis Assays

For proliferation, 10 μM BrdU was added to INS-1E cells for 3 h, and to rat or human islets for 24 h. Proliferation was estimated using an immunohistochemical assay kit as described by the manufacturer (BrdU labeling and detection Kit, Roche Diagnostics, Rotkreuz, Switzerland). Cell apoptosis induced either by cytokines or streptozotocin was measured using the TUNEL assay (In Situ Cell Death Detection Kit, Roche). Alternatively, the Cell Death Detection ELISA^{PLUS} (Roche) was used to quantify the degree of cytoplasmic histone-associated-DNA-fragments. Results of BrdU and TUNEL assays are expressed as a percentage of BrdU- or TUNEL-positive cells (fluorescein- or TMR-red-labelled nuclei) over the total amount of cells (nuclei staining by DAPI). Results of ELISA are presented as a percentage of apoptotic enrichment compared to untreated islets.

Toxicity Test

The MTT (C,N-diphenyl-N′-4,5-dimethyl thiazol-2-yl tetrazolium bromide) colorimetric assay (reduction of tetrazolium salt to formazan) was used to measure cell viability. The assay provides an indirect estimation of mitochondrial oxidative processes of living cells (Janjic, 1992). Briefly, 40 isolated human islets were incubated for 24 or 48 hours with DMSO or a combination of DMSO and the LRH-1 agonist of Formula II. Islets were washed and pre-incubated with KRBH at 37° C. for 40 minutes. Islets were then incubated at 37° C. for 90 minutes with KRBH containing yellow MTT (0.5 mg/ml). Cells were wash and incubated for 20 minutes at room temperature in DMSO to solubilize formazan crystals. Colorimetric measurements were performed at 570 nm with reference wavelength 650 nm automatically subtracted.

Glucose-Stimulated Insulin Secretion

Twenty human islets/sample were preincubated with KRBH buffer (135 mM NaCl, 3.6 mM KCl, 2 mM NaHCO₃, 0.5 mM NaH₂PO₄, 0.5 mM MgSO₄, 1.5 mM CaCl₂, 10 mM HEPES, pH 7.4, and 0.1% bovine serum albumin) containing 2.5 mM glucose for 30 min. Cells were then incubated for 30 min in KRBH buffer containing 2.5 mM or 16.5 mM glucose. In some instances islets were also cultured in the presence of increasing concentrations of a LRH-1 agonist of Formula II or glibenclamide. Insulin content, obtained by 10% acetic acid/ethanol (v/v) treatment on islets, and secreted insulin were quantified using the rat insulin enzyme immunoassay kit (Spi-Bio, Montigny le Bretonneux, France).

DNA Assay

Trypsinized islets were incubated in NaCl—PO4 solution and sonicated. After a 30-minute incubation, 900 μl bisbenzimide was added to 100 μl of DNA sample and the mixture was analysed by fluorimetry at 356 nm. (Labarca, 1980).

Statistical Analysis

Results are expressed as mean+/−SE. Where indicated, the statistical significance of the differences between groups was estimated by Student's t-test. * and ** indicate statistical significance with p<0.05 and p<0.01, respectively.

### EXAMPLE 1

### LRH-1 is Expressed in Pancreatic Islet β-Cells

Earlier in situ hybridization studies have suggested that LRH-1 expression is confined exclusively to the exocrine compartment in adult mouse pancreas (Fayard, 2003; Rausa, 1999). In contrast, a more recent study has shown that the LRH-1 transcript is detected in the human endocrine pancreas (Chuang, 2008). In order to clarify discrepancies and to determine whether LRH-1 is expressed in pancreatic β-cells quantitative RT-PCR and immunofluorescent studies on rat and human islets were conducted. It was found that LRH-1 was expressed in mature rodent and human islets of Langerhans as well as in FACS purified β- and non β-cells of these islets, albeit at lower levels than those detected in the control liver and intestine samples (FIG. 1A). LRH-1 transcript levels appeared more abundant in the non β-cell fraction as compared to purified β-cells. Interestingly, rat and human islets expressed similar levels of the nuclear orphan receptor whereas transcript levels were 6-fold higher in rat liver as compared to the human organ. Co-immunofluorescence analysis performed using anti-LRH-1 and anti-insulin serum confirmed the presence of endogenous LRH-1 in β- as well as non β-cells (FIG. 1B). In rat islet, the staining was dispersed throughout the nuclei and cytoplasm whereas in human islet LRH-1 predominantly stained in sub-regions of the nuclei. These results clearly demonstrate that the nuclear receptor is expressed in rodent and human pancreatic islets, including β-cells.

### EXAMPLE 2

### Adenoviral-Mediated Over Expression of LRH-1 in Either Islets or INS-1E Cells does not Alter Proliferation

In the attempt to gain insight into the functional impact of LRH-1 on β-cells, the transcription factor was conditionally over expressed in islets using tetracycline inducible adenoviruses. Isolated human islets were infected with Ad-hLRH-1 and incubated for 48 h in the presence or absence of increasing concentration of the inducing tetracycline analogue, doxycycline. Quantitative RT-PCR established a dose-dependent stimulation in LRH-1 expression levels, reaching a 48-fold increase as compared to control untreated islets at 1 μg/ml doxycycline (FIG. 2A). A strong nuclear immunostaining was detected in 80% of human islet cells treated with doxycycline while a diffused nuclear and cytoplasmic staining was observed in control islets (FIG. 2B). Similar results were obtained in rat islets as well as in the insulinoma INS-1E cell line (Data not shown). As LRH-1 was previously shown to induce cell replication of pancreatic LTPA and hepatic FL83B cells (Botrugno, 2004), we next investigated the impact of LRH-1 over expression on islet cell proliferation. Over expression of LRH-1 did not alter cell proliferation in INS-1E cells, rat islets or human islets as compared to matched control (FIG. 3).

### EXAMPLE 3

### Rat and Human Islets Over Expressing LRH-1 are Protected Against Cytokine and Streptozotocin-Induced Apoptosis

As LRH-1 expression confers anti-apoptotic properties to the hepatocellular carcinoma cell line BEL-7402 (Wang, 2005) and inhibits cytokine-elicited inflammatory responses in the liver (Venteclef, 2006), the potential protective role of LRH-1 over expression in rat and human islets exposed to either cytokines or streptozotocin was evaluated. A 4-fold increase in TUNEL-positive cells was estimated in control rat islets cultured in the presence of cytokines or streptozotocin. However, doxycycline-induced LRH-1 expression completely protected islets against apoptosis (FIG. 4). Similarly, human islets exhibited a 10-fold increase in cytokine-mediated apoptosis, an effect that was dose dependently attenuated by increasing concentrations of doxycycline (FIG. 5A). LRH-1 over expressing human islets were also refractory to apoptosis induced by elevated doses of streptozotocin (FIG. 5B). As the TUNEL assay may be prone to false-positive results (Galluzzi, 2009), we alternatively measured apoptosis using a cell death detection ELISA system and found identical results (FIG. 5C). Taken together these results clearly suggest that LRH-1 is involved in cell survival rather than cell proliferation in the endocrine pancreas.

### EXAMPLE 4

### The Synthetic LRH-1 Agonists are not Toxic to Islets and Activates the Nuclear Orphan Receptor

Recently, small non-polar bicyclic compounds were identified as potent ligands for LRH-1 in human hepatic cells (Whitby, 2006). Thus a derivative compound (Formula II) was synthesised, which possess a slightly increased binding activity as well as efficacy for LRH-1 and determined whether this one could sufficiently stimulate the activity of the endogenous nuclear receptor to protect islets against harmful environmental insults. We initially, established whether increasing concentrations of BL001 or DMSO, the vehicle, were toxic to islet cells. Assessment of viability by MTT revealed no apparent detrimental effect of either 0.5% DMSO or 50 uM of a LRH-1 agonist of Formula II at 24 or 48 hours post treatment (FIG. 6). Transcript levels for LRH-1 or its downstream target gene, shp were then estimated at various time intervals in human islets treated with 17β-estradiol, a LRH-1 agonist of Formula II or a combination of both. Estrogens were previously shown to induce LRH-1 expression in the breast cancer MCF7 cell line within 6 hours post treatment (Annicotte, 2005). Consistent with the latter, LRH-1 expression was transiently increased in human islets 6 hours after addition of 10 nM 17β-estradiol while 10 or 50 uM a LRH-1 agonist of Formula II did not enhance transcript levels of the nuclear receptor at any time point (FIG. 7A). Interestingly, the addition of a LRH-1 agonist of Formula II to 17β-estradiol prevented the induction of LRH-1 expression observed in hormone-treated islets. In contrast, SHP transcript levels were transiently increased 2-fold in islets treated with a LRH-1 agonist of Formula II for 10 hours (FIG. 7B). Increasing the concentration of a LRH-1 agonist of Formula II or the addition of 17β-estradiol did not further stimulate expression of SHP. Thus taken together these results clearly show that LRH-1 agonists stimulates endogenous LRH-1 activity rather than expression, which then enhances expression of the downstream target gene shp.

### EXAMPLE 5

### LRH-1 Agonists Protect Human Islets from Cytokine-Induced Apoptosis

The capacity of LRH-1 agonists to preserve islet cells against stress-induced apoptosis was next evaluated. To this end, human islets were treated with 4 consecutive doses of either 10 uM of a LRH-1 agonist of Formula II or 0.1% DMSO at intervals of 24 hours (−24, 0, 24 and 48 hours). In addition, islets were also treated with 3 doses of cytokines starting 25 hours after the initial drug treatment (1, 25 and 49 hours). Apoptosis was then measured at 24 and 48 hours (FIG. 8A). Cytokines-treated islets exhibited 160% enrichment in apoptotic cells as compared to control untreated islets 24 hours post-treatment (FIG. 8B). Remarkably this effect was completely abrogated in islets cultured in the presence of 10 uM of a LRH-1 agonist of Formula II (FIG. 8B). A similar beneficial effect was observed in cytokines-treated islets at 72 hours post treatment (FIG. 8C). Taken together, these results demonstrate that activation of endogenous LRH-1 using a novel agonist can confer protection against cytokine-induced apoptosis to human islets.

### EXAMPLE 6

### LRH-1 Agonists Stimulate Insulin Secretion in Human Normal and Diabetic Islets

Insulin secretion in response to a LRH-1 agonist in the presence of either 2 mM or 16.5 mM glucose was then studied in control and Type 2 diabetic islets. Both basal and glucose-induced insulin secretion exhibited little variation in control islets cultured in the presence of increasing percentage of DMSO (FIG. 9A). Interestingly, although glucose-induced insulin secretion was unchanged in the presence of a LRH-1 agonist of Formula II, the highest concentration (50 uM) resulted in a 50% increase in basal insulin exocytosis (FIG. 9A). In contrast to control islets, islets isolated from a Type 2 diabetic donor and cultured in the presence of DMSO were refractory to glucose-induced insulin secretion, substantiating a β-cell dysfunction associated with Type 2 diabetes (FIG. 9B). Although not significant, a slight increase in glucose-induced insulin secretion was observed in islets treated with 50 uM of the LRH-1 agonist of Formula II. As expected, the sulfonylurea derivative, Glibenclamide, prompted an 80% increase in insulin secretion at low glucose. This effect was further accentuated at high glucose reaching 214% as compared to control islets (FIG. 9B). Islets isolated from a second Type 2 diabetic donor that were cultured in the presence of a LRH-1 agonist of Formula II not only displayed increased insulin secretion in response to high glucose concentrations but also to low glucose concentrations (FIG. 9C). Islet viability was also estimated in the first donor and found slightly reduced at 50 uM of a LRH-1 agonist of Formula II (FIG. 9D). However, this effect appears to be mediated by DMSO, as increasing the concentration of the vehicle alone resulted in decreased viability. Consistent with a previous report, glibenclamide also impaired cell viability, which could ultimately lead to apoptosis (Maedler, 2005). Thus, similar to glibenclamide, LRH-1 agonists appears to stimulate insulin secretion in diabetic islets both at low and high glucose concentrations.

### LIST OF CITED REFERENCES

Annicotte et al. (2005), Oncogene 24, 8167-8175
Annicotte et al. (2003), Mol Cell Biol 23, 6713-6724
Asfari et al. (1992), Endocrinology 130, 167-178
Boerboom et al. (2000), Endocrinology 141, 4647-4656
Botrugno et al. (2004), Mol Cell 15, 499-509
Brun et al. (2004), J Cell Biol 167, 1123-1135
Calcutt et al. (2009), Nat Rev Drug Discov 8, 417-429
Chuang et al. (2008), Mol Endocrinol 22, 2353-2363
Contreras et al. (2002), Transplantation 74, 1252-1259
Fayard et al. (2003), J Biol Chem 278, 35725-35731
Galarneau et al. (1996), Mol Cell Biol 16, 3853-3865
Galluzzi et al. (2009), Cell Death Differ 16, 1093-1107
Gauthier et al. (2004), J Biol Chem 279, 31121-31130
Janjic and Wollheim (1992), Diabetologia 35, 482-485
Labarca and Paigen (1980), Anal Biochem 102, 344-352
Le May et al. (2006), Proc Natl Acad Sci USA 103, 9232-9237
Lee and Moore (2008), Front Biosci 13, 5950-5958
Maedler et al. (2005), J Clin Endocrinol Metab 90, 501-506
Merglen et al. (2004), Endocrinology 145, 667-678
Ortlund et al. (2005), Nat Struct Mol Biol 12, 357-363
Pare et al. (2001), J Biol Chem 276, 13136-13144
Parnaud et., (2008) Diabetologia 51, 91-100
Rausa et al. (1997), Developmental Biology 92, 228-246
Rausa et al. (1999), Mechan Devel 89, 185-188
Roglic et al. (2005), Diabetes Care 28, 2130-2135
Venteclef et al. (2006), Mol Cell Biol 26, 6799-6807
Wang et al. (2005), Biochem Biophys Res Commun 333, 917-924
Whitby et al. (2006), J Med Chem 49, 6652-6655
Whitby et al. (2011), J Med Chem 54, 2266-2281

1.-16. (canceled)

17. A method of preventing progressive loss of pancreatic β-cells the method comprising contacting pancreatic β-cells will an LRH-1 agonist.

18. The method of claim 17, wherein the pancreatic β-cells are pancreatic islet β-cells.

19. The method of claim 17, wherein the progressive loss of pancreatic β-cells is due to cell death of pancreatic islet β-cells.

20. The method of claim 17, wherein cell death is due to apoptosis.

21. The method of claim 20, wherein apoptosis is stress-induced.

22. The method of claim 21, wherein the stress-induced apoptosis is due to pro-inflammatory cytokines.

23. The method of claim 17, wherein contacting the pancreatic β-cells with the LRH-1 agonist results in preservation or restoration of pancreatic β-cells.

24. The method of claim 17, wherein contacting the pancreatic β-cells with the LRH-1 agonists results in an increment of survival of pancreatic β-cells.

25. The method of claim 17, wherein contacting the pancreatic β-cells with the LRH-1 results in the increment of the performance of pancreatic β-cells, preferably pancreatic islet β-cells.

26. The method of claim 17, wherein contacting the pancreatic β-cells with the LRH-1 agonist results in an the increment of the survival of a β-cell graft.

27. The method of claim 26, wherein the β-cell graft is a pancreatic β-cell graft or a pancreatic islet β-cell graft.

28. The method of claim 17, wherein the method is used in the in vitro preservation of pancreatic β-cells.

29. The method of claim 17, wherein the method is used in a method of transplanting pancreatic islet cells comprising:
(a) isolating pancreatic islet cells from a donor;
(b) cultivating said cells in the presence of an LRH-1 agonist; and
(c) transplanting said cells into a subject,

30. The method of claim 17, wherein contacting the pancreatic β-cells with the LRH-1 agonists results in maintaining insulin secretion.

31. The method of claim 17, wherein the LRH-1 agonist is a 9-substituted bicycle [3.3.0] octane derivative of formula (Ia) 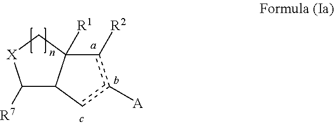
wherein
A is methyl, ethyl, aryl, preferably phenyl, cycloalkyl, preferably cyclohexyl or 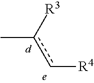
X is C(R)₂ or NR;
n is 1 or 2;
R is H, alkyl or R is OR¹⁰ wherein R¹⁰ is H, alkyl, acyl;
R¹ is —N(R⁵)₂, OR¹¹ or C(R¹²)═CH₂;
R² is H, alkyl, halogen, alkenyl, alkynyl, aryl, aralkyl, aralkenyl, alkylene, alkenylene, cycloalkyl, cycloalkylene or N-heterocyclyl which can be optionally substituted;
R³ and R⁴ are independently H, alkyl, alkenyl, alkynyl, aryl, aralkyl, aralkenyl, alkylene, alkenylene, cycloalkyl, cycloalkylene, halogen or N-heterocyclyl;
each R⁵ is independently hydrogen, alkyl, alkenyl, alkynyl, aryl, aralkyl or aralkenyl;
R⁷ is H, OH, OR⁸ wherein R⁸ is alkyl, acyl or aryl; and
R¹¹ is C₂-C₄ optionally substituted C₂-C₄ alkyl, optionally substituted aralkyl, optionally substituted cycloalkyl;
R¹² is optionally substituted aryl or R¹² is C₂-C₄ alkyl;
wherein 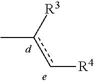
when A is
there is independently a maximum of one double bond between each of the carbon atoms of the centers a-b and b-c and d-e
and when A is methyl, aryl or cycloalkyl
there is independently a maximum of one double bond between each of the carbon atoms of the centers a-b and b-c.

32. The method of claim 31, wherein said LRH-1 agonist is a 9-substituted bicycle [3.3.0] octane derivative having formula I 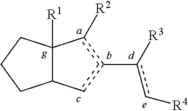
wherein
R¹ is —N(R⁵)₂,
R² is H, alkyl, halogen, alkenyl, alkynyl, aryl, aralkyl, aralkenyl, alkylene, alkenylene, cycloalkyl, cycloalkylene or N-heterocyclyl which can be optionally substituted
R³ and R⁴ are independently alkyl, alkenyl, alkynyl, aryl, aralkyl, aralkenyl, alkylene, alkenylene, cycloalkyl, cycloalkylene, halogen or N-heterocyclyl;
each R⁵ is independently hydrogen, alkyl, alkenyl, alkynyl, aryl, aralkyl or aralkenyl;
and
each R⁶ is a straight or branched alkylene chain optionally substituted by hydroxy, mercapto, alkylthio, aryl, cycloalkyl, —N(R⁵)₂, —C(O)OR⁵ or —C(O)N(R⁵)₂;
and
wherein there is independently a maximum of one double bond between each of the carbon atoms of the centers a-b and b-c and d-e.

33. A method of preventing or treating of type I diabetes, the method comprising contacting pancreatic β-cells will an LRH-1 agonist.
